SEQUENCE LISTING (91 sequences)

SEQ ID NO: 1 - DNA, 1665 bp; single-stranded, linear
  CDS 15..1649
gaattcggcttcatatggcggctaaagacgtaaaattcggtaacgacgctcgtgtgaaaatgctgcgcggcgtaaacgtactggcagatgcagtgaaagttaccctcggcccaaaaggccgtaacgtagttctggataaatctttcggtgcaccgaccatcactaaagatggtgtttccgtagcacgtgaaatcgaactggaagacaagttcgaaaacatgggtgcgcagatggtgaaagaagttgcctctaaagcgaacgacgctgcaggtgacggtaccaccaccgcaaccgtactggctcagtccatcatcactgaaggcctgaaagccgttgctgcgggcatgaacccgatggatctgaaacgtggtatcgacaaagctgtcgctgctgctgttgaagaactgaaagcactgtccgtaccgtgctccgactctaaagctattgctcaggttggtaccatctccgctaactccgacgaaaccgtaggtaaactgatcgctgaagcgatggacaaagtcggtaaagaaggcgtgatcaccgttgaagacggtaccggtctgcaggacgaactggacgtggttgaaggtatgcagttcgaccgtggctacctgtctccttacttcatcaacaagccggaaactggcgcagtagaattggaaagcccgttcatcctgctggctgacaagaaaatctccaacatccgcgaaatgctgccggttctggaagctgtagcgaaagcaggcaaaccgctgctgatcatcgctgaagatgttgaaggcgaagcgctggcaactctggttgttaacaccatgcgcggtatcgtaaaagtcgctgcggttaaagcacctggcttcggcgatcgtcgtaaagcaatgctgcaggatatcgctaccctgaccggtggtaccgttatctctgaagagatcggtatggagctggaaaaagcaactctggaagatctgggccaggcgaaacgcgttgttatcaacaaagataccaccaccatcatcgatggcgtgggcgacgaagctgcaatccagggtcgcgtgactcagattcgtcagcagatcgaagaagcaacttccgactatgaccgtgaaaaactgcaggagcgcgtagcgaaactggcaggcggcgttgcggttatcaaagttggtgctgcgactgaagttgaaatgaaagagaagaaagcccgcgttgaagatgccctgcacgctacccgtgctgcggtagaagaaggcgtggttgctggtggtggcgttgcgctgattcgcgtagcgtctaaaattgccggcctgaaaggtcagaacgaagaccagaacgtaggtatcaaagttgcgctgcgcgcaatggaatccccactgcgtcaaatcgtactgaactgcggcgaagagccgtctgtagtggctaacaccgtgaaagccggtgacggtaactacggttacaacgctgcaactgaagaatacggcaacatgatcgacatgggtatcctggatccaaccaaagtaactcgttctgctctgcagtacgcggcttctgttgcgggtctgatgatcaccaccgagtgcatggttaccgacctgccgaaaggcgatgcacctgacttaggtgctgctggtggtatgggcggcatgggcggaatgatgtgatcaagccgaattc

SEQ ID NO: 2 - protein, 545 aa
MAAKDVKFGNDARVKMLRGVNVLADAVKVTLGPKGRNVVLDKSFGAPTITKDGVSVAREIELEDKFENMGAQMVKEVASKANDAAGDGTTTATVLAQSIITEGLKAVAAGMNPMDLKRGIDKAVAAAVEELKALSVPCSDSKAIAQVGTISANSDETVGKLIAEAMDKVGKEGVITVEDGTGLQDELDVVEGMQFDRGYLSPYFINKPETGAVELESPFILLADKKISNIREMLPVLEAVAKAGKPLLIIAEDVEGEALATLVVNTMRGIVKVAAVKAPGFGDRRKAMLQDIATLTGGTVISEEIGMELEKATLEDLGQAKRVVINKDTTTIIDGVGDEAAIQGRVTQIRQQIEEATSDYDREKLQERVAKLAGGVAVIKVGAATEVEMKEKKARVEDALHATRAAVEEGVVAGGGVALIRVASKIAGLKGQNEDQNVGIKVALRAMESPLRQIVLNCGEEPSVVANTVKAGDGNYGYNAATEEYGNMIDMGILDPTKVTRSALQYAASVAGLMITTECMVTDLPKGDAPDLGAAGGMGGMGGMM

SEQ ID NO: 3 - DNA, 1654 bp; single-stranded, linear
  CDS 15..1637
gaattcggcttcatatggcaaaagaaattaaattttcatcagatgcccgttcagctatggtccgtggtgtcgatatccttgcagatactgttaaagtaactttgggaccaaaaggtcgcaatgtcgttcttgaaaagtcattcggttcacccttgattaccaatgacggtgtgactattgccaaagaaattgaattagaagaccattttgaaaatatgggtgccaaattggtatcagaagtagcttcaaaaaccaatgatatcgcaggtgatggaactacaactgcaactgttttgacccaagcaatcgtccgtgaaggaatcaaaaacgtcacagcaggtgcaaatccaatcggtattcgtcgtgggattgaaacagcagttgccgcagcagttgaagctttgaaaaacaacgtcatccctgttgccaataaagaagctatcgctcaagttgcagccgtatcttctcgttctgaaaaagttggtgagtacatctctgaagcaatggaaaaagttggcaaagacggtgtcatcaccatcgaagagtcacgtggtatggaaacagagcttgaagtcgtagaaggaatgcagtttgaccgtggttacctttcacagtacatggtgacagatagcgaaaaaatggtggctgaccttgaaaatccgtacattttgattacagacaagaaaatttccaatatccaagaaatcttgccacttttggaaagcattctccaaagcaatcgtccactcttgattattgcggatgatgtggatggtgaggctcttccaactcttgttttgaacaagattcgtggaaccttcaacgtagtagcagtcaaggcacctggttttggtgaccgtcgcaaagccatgcttgaagatatcgccatcttaacaggcggaacagttatcacagaagaccttggtcttgagttgaaagatgcgacaattgaagctcttggtcaagcagcgagagtgaccgtggacaaagatagcacggttattgtagaaggtgcaggaaatcctgaagcgatttctcaccgtgttgcggttatcaagtctcaaatcgaaactacaacttctgaatttgaccgtgaaaaattgcaagaacgcttggccaaattgtcaggtggtgtagcggttattaaggtcggagccgcaactgaaactgagttgaaagaaatgaaactccgcattgaagatgccctcaacgctactcgtgcagctgttgaagaaggtattgttgcaggtggtggaacagctcttgccaatgtgattccagctgttgctaccttggaattgacaggagatgaagcaacaggacgtaatattgttctccgtgctttggaagaacctgttcgtcaaattgctcacaatgcaggatttgaaggatctatcgttatcgatcgtttgaaaaatgctgagcttggtataggattcaacgcagcaactggcgagtgggttaacatgattgatcaaggtatcattgatccagttaaagtgagtcgttcagccctacaaaatgcagcatctgtagccagcttgattttgacaacagaagcagtcgtagccaataaaccagaaccagtagccccagctccagcaatggatccaagtatgatgggtggaatgggcggatgatcaaagccgaattc

SEQ ID NO: 4 - protein, 541 aa
MAKEIKFSSDARSAMVRGVDILADTVKVTLGPKGRNVVLEKSFGSPLITNDGVTIAKEIELEDHFENMGAKLVSEVASKTNDIAGDGTTTATVLTQAIVREGIKNVTAGANPIGIRRGIETAVAAAVEALKNNVIPVANKEAIAQVAAVSSRSEKVGEYISEAMEKVGKDGVITIEESRGMETELEVVEGMQFDRGYLSQYMVTDSEKMVADLENPYILITDKKISNIQEILPLLESILQSNRPLLIIADDVDGEALPTLVLNKIRGTFNVVAVKAPGFGDRRKAMLEDIAILTGGTVITEDLGLELKDATIEALGQAARVTVDKDSTVIVEGAGNPEAISHRVAVIKSQIETTTSEFDREKLQERLAKLSGGVAVIKVGAATETELKEMKLRIEDALNATRAAVEEGIVAGGGTALANVIPAVATLELTGDEATGRNIVLRALEEPVRQIAHNAGFEGSIVIDRLKNAELGIGFNAATGEWVNMIDQGIIDPVKVSRSALQNAASVASLILTTEAVVANKPEPVAPAPAMDPSMMGGMGG

SEQ ID NO: 5 - DNA, 1662 bp; single-stranded, linear
  CDS 15..1646
gaattcggcttcatatggcggctaaagatgtaaaattcggtaacgacgctcgtgtaaaaatgctccgcggcgtaaacgtactggcagacgcagttaaagtaaccctgggcccgaaaggccgtaacgtagtgctggacaaatccttcggcgcgccaaccatcacgaaagatggtgtttctgtagcacgtgaaatcgagctggaagacaagttcgaaaacatgggcgcgcagatggtgaaagaagtggcctctaaagcgaacgacgctgcaggcgacggtaccaccaccgcgaccgtgctggctcaggctatcatcaccgaaggtctgaaagccgttgctgcgggcatgaacccaatggatctgaaacgtggtatcgacaaagctgtcgcgtccgctgttgaagaactgaaagcgctgtccgtaccgtgctctgactctaaagccattgctcaggtaggtaccatctccgctaactccgacgaaaccgtaggtaaactgatcgcggaagcgatggataaagtcggtaaagaaggcgtgatcaccgttgaagacggtaccggtctggaagacgaactggacgtggttgaaggtatgcagttcgaccgcggttacctgtccccatacttcatcaacaagccagaaactggcgctgttgagctggaaagcccgttcatcctgctggctgacaagaaaatctccaacatccgcgaaatgctgccagtgctggaagccgttgcgaaagcaggcaaaccgctggttatcattgctgaagacgttgaaggcgaagcgctggcgaccctggtggttaacaccatgcgtggcatcgtgaaagtggctgcggttaaagcacctggcttcggcgaccgccgtaaagcgatgctgcaggatatcgctaccctgaccggcggtaccgtcatctctgaagagatcggtatggagctggaaaaagcgaccctggaagacctgggccaggctaaacgtgttgtgatcaacaaagacaccaccaccatcatcgatggcgtgggcgacgaagcggcgattcagggccgtgttggtcagatccgtaagcagatcgaagaagccacttccgattacgaccgtgaaaaactgcaggagcgcgtagcgaaactggcaggcggtgttgcggtaatcaaagtcggtgctgcgactgaagttgaaatgaaagagaaaaaagcacgcgttgacgatgccctgcacgcgacccgtgctgcggtagaagaaggcgtggttgctggtggtggtgtggcgctggtgcgtgttgccgcgaaactgtccggcctgactgctcagaacgaagatcagaacgtgggtatcaaagttgcgctgcgcgcaatggaagctccactgcgtcagatcgtgtccaacgccggtgaagagccatctgttgtgaccaacaacgtgaaagcaggcgaaggtaactacggttacaacgcagcaactgaagaatacggcaacatgatcgacttcggtatcctggatccaaccaaagtgacccgttctgctctgcagtacgcggcatctgtcgctggcctgatgatcaccaccgagtgcatggtgaccgacctgcctaaaggcgacgcacctgacttaggtgctgcaggcatgggtgggatgggcggtatgatgtgatcaagccgaattc

SEQ ID NO: 6 - protein, 544 aa
MAAKDVKFGNDARVKMLRGVNVLADAVKVTLGPKGRNVVLDKSFGAPTITKDGVSVAREIELEDKFENMGAQMVKEVASKANDAAGDGTTTATVLAQAIITEGLKAVAAGMNPMDLKRGIDKAVASAVEELKALSVPCSDSKAIAQVGTISANSDETVGKLIAEAMDKVGKEGVITVEDGTGLEDELDVVEGMQFDRGYLSPYFINKPETGAVELESPFILLADKKISNIREMLPVLEAVAKAGKPLVIIAEDVEGEALATLVVNTMRGIVKVAAVKAPGFGDRRKAMLQDIATLTGGTVISEEIGMELEKATLEDLGQAKRVVINKDTTTIIDGVGDEAAIQGRVGQIRKQIEEATSDYDREKLQERVAKLAGGVAVIKVGAATEVEMKEKKARVDDALHATRAAVEEGVVAGGGVALVRVAAKLSGLTAQNEDQNVGIKVALRAMEAPLRQIVSNAGEEPSVVTNNVKAGEGNYGYNAATEEYGNMIDFGILDPTKVTRSALQYAASVAGLMITTECMVTDLPKGDAPDLGAAGMGGMGGMM

SEQ ID NO: 7 - DNA, 1661 bp; single-stranded, linear
  CDS 15..1649
gaattcggcttcatatggcaaaagaaatcaaattttcagcagatgcgcgtgctgccatggtgcgcggagttgatatgttagcagataccgtcaaagtaacgcttggtcctaaagggcgcaatgttgttcttgaaaaagcttttggttctcccttaattactaatgacggggtaaccattgctaaagagatcgaattagaagatcattttgaaaacatgggagcaaaattggtgtctgaagtggcttctaaaaccaatgatattgctggtgatgggacgactactgcaacagttttgacacaagccattgttcatgaaggactaaaaaatgtgacagcaggtgctaatccaattggtatccgtcgaggcattgaaacagcaacagcaacagctgttgaagccttgaaagccattgctcaacctgtatctggcaaggaagctattgctcaggtcgctgcagtatcatcacgctctgaaaaagttggagagtatatctcagaagctatggagcgtgtgggcaacgatggtgtgattaccatcgaagaatctcgaggtatggaaacagaacttgaagtggttgaaggcatgcaatttgaccgtggttacctgtctcaatacatggtcacagacaatgaaaaaatggttgcagaccttgaaaacccatttatcttaatcacggataaaaaagtgtcaaacatccaagacattttgccactacttgaggaagttcttaaaaccaaccgtccattactcattattgcagatgatgtggatggtgaagcacttccaacccttgtcttgaacaagattcgtggtactttcaatgtggttgctgtcaaagcgccaggatttggtgatcgtcgtaaagctatgcttgaagacattgctatcttgacaggtggtacagtgattacagaggatctaggacttgaattaaaagatgctacaatgacagcccttggacaggctgctaagattacagttgataaagatagcacagtaattgttgaaggttcaggaagttcagaagctattgctaaccgtattgcactgattaaatcgcaattagaaacaacaacttctgactttgaccgtgaaaaactacaagaacgtttggcgaaattagctggtggtgtagctgttatcaaagtaggagctccaacagagacagctttaaaagaaatgaaacttcgcattgaggatgctctaaatgctacacgtgcagccgttgaagaaggtatcgttgctggtggtggaacagcacttattacggttattgaaaaagtagcagctcttgagcttgagggcgatgatgctactggacgtaacattgtgcttcgtgctctagaagagcctgtacgtcaaattgctttaaatgctgggtacgaaggctccgtagttattgacaagttgaaaaacagccctgcaggaacaggatttaatgctgcaacaggtgagtgggttgatatgattaaaacaggaatcattgaccctgtcaaagtaacacgatcagcgcttcaaaatgcagcttctgtagctagtcttattttgacaacagaagcagttgttgctaataaacctgaaccagctacgccagcgccagcaatgccagcaggtatggatccaggaatgatgggtgggatgggcggataagccgaattc

SEQ ID NO: 8 - protein, 545 aa
MAKEIKFSADARAAMVRGVDMLADTVKVTLGPKGRNVVLEKAFGSPLITNDGVTIAKEIELEDHFENMGAKLVSEVASKTNDIAGDGTTTATVLTQAIVHEGLKNVTAGANPIGIRRGIETATATAVEALKAIAQPVSGKEAIAQVAAVSSRSEKVGEYISEAMERVGNDGVITIEESRGMETELEVVEGMQFDRGYLSQYMVTDNEKMVADLENPFILITDKKVSNIQDILPLLEEVLKTNRPLLIIADDVDGEALPTLVLNKIRGTFNVVAVKAPGFGDRRKAMLEDIAILTGGTVITEDLGLELKDATMTALGQAAKITVDKDSTVIVEGSGSSEAIANRIALIKSQLETTTSDFDREKLQERLAKLAGGVAVIKVGAPTETALKEMKLRIEDALNATRAAVEEGIVAGGGTALITVIEKVAALELEGDDATGRNIVLRALEEPVRQIALNAGYEGSVVIDKLKNSPAGTGFNAATGEWVDMIKTGIIDPVKVTRSALQNAASVASLILTTEAVVANKPEPATPAPAMPAGMDPGMMGGMGG

SEQ ID NO: 9 - protein, 544 aa
MAKEIKFSEEARRAMLRGVDALADAVKVTLGPKGRNVVLEKKFGSPLITNDGVTIAKEIELEDAFENMGAKLVAEVASKTNDVAGDGTTTATVLAQAMIREGLKNVTAGANPVGVRKGMEQAVAVAIENLKEISKPIEGKESIAQVAAISAADEEVGSLIAEAMERVGNDGVITIEESKGFTTELEVVEGMQFDRGYASPYMVTDSDKMEAVLDNPYILITDKKITNIQEILPVLEQVVQQGKPLLLIAEDVEGEALATLVVNKLRGTFNAVAVKAPGFGDRRKAMLEDIAVLTGGEVITEDLGLDLKSTQIAQLGRASKVVVTKENTTIVEGAGETDKISARVTQIRAQVEETTSEFDREKLQERLAKLAGGVAVIKVGAATETELKERKLRIEDALNSTRAAVEEGIVSGGGTALVNVYNKVAAVEAEGDAQTGINIVLRALEEPIRQIAHNAGLEGSVIVERLKNEEIGVGFNAATGEWVNMIEKGIVDPTKVTRSALQNAASVAAMFLTTEAVVADKPEENGGGAGMPDMGGMGGMGGMM

SEQ ID NO: 10 - protein, 539 aa
MAKTLLFGEEARRSMQAGVDKLANTVKVTLGPKGRNVILDKKFGSPLITNDGVTIAREIELEDAYENMGAQLVKEVATKTNDVAGDGTTTATLLAQAIIREGLKNVTAGANPILIRNGIKTAVEKAVEEIQKISKPVNGKEDIARVAAISAADEKIGKLIADAMEKVGNEGVITVEESKSMGTELDVVEGMQFDRGYVSAYMVTDTEKMEAVLDNPLVLITDKKISNIQDLLPLLEQIVQAGKKLLIIADDIEGEAMTTLVVNKLRGTFTCVGVKAPGFGDRRKEMLQDIATLTGGVVISDEVGGDLKEATLDMLGEAESVKVTKESTTIVNGRGNSEEIKNRINQIKLQLEATTSEFDKEKLQERLAKLAGGVAVVKVGAATETELKESKLRIEDALAATKAAVEEGIVPGGGTAYVNVINEVAKLTSDIQDEQVGINIIVRSLEEPMRQIAHNAGLEGSVIIEKVKNSDAGVGFDALRGEYKDMIKAGIVDPTKVTRSALQNAASVASTFLTTEAAVADIPEKEMPQGAGMGMDGMY

SEQ ID NO: 11 - protein, 551 aa
MANMVVTGEQLDKSIREVVRILEDAVGCTAGPKGLTVAISKPYGAPEVTKDGYKVMKSIKPEDPLALAIANIIAQSASQCNDKVGDGTTTCSILTAKVIEEVSKVKAAGADIICVREGVLKAKEAVLEALKCMKREVLSEEEIAQVATISANGDKNIGTKIAQCVKEVGKDGVITVEESKGFKELDVEKTDGMQFDRGYLSPYFVTNSEKMLVEFENPYILLTEKKLNIIQPLLPILENIARSGRPLLIIAEDVEGEALSTLVLNKLRGGLHVAAVKAPGFGDRRKDMLGDIAILTGAKHVINDELAIKMEDLTLCDLGTAKNIRITKDTTTIIGSVDNSCAHVQSRICQIRMQIDNSTSDYDKEKLQERLAKLSGGVAVLKVGGSSEVEVKERKDRVEDALHATRAAVEEGVVPGGGAALLYTLSALDNLKSKNDDEQLGINIVKRALQAPIKRIIKNAGSENAPCVIAHLLKQNDKELIFNVDVTNFANAFTSGVIDPLKVVRIAFDFAVSLAAVFMTLNAIVVDIPSKDDNSAAGGAGMGGMGGMGGF

SEQ ID NO: 12 - protein, 548 aa
MAAKDVKFGNDARVKMLNGVNILADAVKVTLGPKGRNVVLDKSFGAPTITKDGVSVAREIELEDKFENMGAQMVKEVASKANDAAGDGTTTATVLAQAIVNEGLKAVAAGMNPMDLKRGIDKAVNSVVAELKNLSKPCETSKEIEQVGTISANSDSIVGQLIAQAMEKVGKEGVITVEDGTGLEDELDVVEGMQFDRGYLSPYFINKPETAGTVELDNPFILLVDKKISNIRELLPVLEAVAKAGKPLLIIAEDVEGEALATLVVNTMRGIVKVAAVKAPGFGDRRKAMLQDIAILTAGTVISEEIGMELEKATLEELGQAKRVVITKDNTTIIDGIGDEAQIKARVVQIRQQIEDSTSDYDKEKLQERVAKLAGGVAVIKVGAATEVAMKEKKDRVDDALHATRAAVEEGIVPGGGVALVRAANKVSATLTGDNEEQNVGIKLALRAMEAPLRQIVENSGEDASVVARDVKDGSGNFGYNATTEEYGDMLEMGILDPTKVTRSALQFAASIAGLMITTECMITDLPKEDKLDAQAAMGGMGGMGGMM

SEQ ID NO: 13 - protein, 549 aa
MAKELRFGDDARLQMLAGVNALADAVQVTMGPRGRNVVLEKSYGAPTVTKDGVSVAKEIEFEHRFMNMGAQMVKEVASKTSDTAGDGTTTATVLARSILVEGHKAVAAGMNPMDLKRGIDKAVLAVTKKLQAMSKPCKDSKAIAQVGTISANSDEAIGAIIAEAMEKVGKEGVITVEDGNGLENELSVVEGMQFDRGYISPYFINNQQNMSCELEHPFILLVDKKVSSIREMLSVLEGVAKSGRPLLIIAEDVEGEALATLVVNNMRGIVKVCAVKAPGFGDRRKAMLQDIAILTKGQVISEEIGKSLEGATLEDLGSAKRIVVTKENTTIIDGEGKATEINARITQIRAQMEETTSDYDREKLQERVAKLAGGVAVIKVGAATEVEMKEKKARVEDALHATRAAVEEGIVAGGGVALIRAQKALDSLKGDNDDQNMGINILRRAIESPMRQIVTNAGYEASVVVNKVAEHKDNYGFNAATGEYGDMVEMGILDPTKVTRMALQNAASVASLMLTTECMVADLPKKEEGVGAGDMGGMGGMGGMGGMMZ

SEQ ID NO: 14 - protein, 541 aa
MAKTIAYDEEARRGLERGLNALADAVKVTLGPKGRNVVLEKKWGAPTITNDGVSIAKEIELEDPYEKIGAELVKEVAKKTDDVAGDGTTTATVLAQALVREGLRNVAAGANPLGLKRGIEKAVEKVTETLLKSAKEVETKDQIAATAAISAGDQSIGDLIAEAMDKVGNEGVITVEESNTFGLQLELTEGMRFDKGYISGYFVTDAERQEAVLEDPFILLVSSKVSTVKDLLPLLEKVIQAGKPLLIIAEDVEGEALSTLVVNKIRGTFKSVAVKAPGFGDRRKAMLQDMAILTGGQVISEEVGLSLESADISLLGKARKVVVTKDETTIVEGAGDSDAIAGRVAQIRTEIENSDSDYDREKLQERLAKLAGGVAVIKAGAATEVELKERKHRIEDAVRNAKAAVEEGIVAGGGVALLHAIPALDELKPEGEEATGANIVRVALERPLKQIAFNGGLEPGVVAEKVRNSPAGTGLNAATGEYEDLLKAGIADPVKVTRSALQNAASIAGLFLTTEAVVADKPEKAAAPAGDPTGGMGGMDF

SEQ ID NO: 15 - protein, 540 aa
MAKTIAYDEEARRGLERGLNALADAVKVTLGPKGRNVVLEKKWGAPTITNDGVSIAKEIELEDPYEKIGAELVKEVAKKTDDVAGDGTTTATVLAQALVREGLRNVAAGANPLGLKRGIEKAVEKVTETLLKGAKEVETKEQIAATAAISAGDQSIGDLIAEAMDKVGNEGVITVEESNTFGLQLELTEGMRFDKGYISGYFVTDPERQEAVLEDPYILLVSSKVSTVKDLLPLLEKVIGAGKPLLIIAEDVEGEALSTLVVNKIRGTFKSVAVKAPGFGDRRKAMLQDMAILTGGQVISEEVGLTLENADLSLLGKARKVVVTKDETTIVEGAGDTDAIAGRVAQIRQEIENSDSDYDREKLQERLAKLAGGVAVIKAGAATEVELKERKHRIEDAVRNAKAAVEEGIVAGGGVTLLQAAPTLDELKLEGDEATGANIVKVALEAPLKQIAFNSGLEPGVVAEKVRNLPAGHGLNAQTGVYEDLLAAGVADPVKVTRSALQNAASIAGLFLTTEAVVADKPEKEKASVPGGGDMGGMDF

SEQ ID NO: 16 - protein, 537 aa
MSKLIEYDETARHAMEVGMNKLADTVRVTLGPRGRHVVLAKAFGGPTITNDGVTVAREIDLEDPFENLGAQLVKSVATKTNDVAGDGTTTATVLAQALVKGGLRMVAAGANPVALGAGISKAADAVSEALLAVATPVAGKDAITQVATVSSRDEQIGALVGEGMNKVGTDGVVSVEESSTLDTELEFTEGVGFDKGFLSAYFVTDFDSQQAVLDDPLVLLHQEKISSLPELLPMLEKVTESGKPLLIVAEDLEGEALATLVVNSIRKTLKAVAVKSPFFGDRRKAFLEDLAIVTGGQVVNPETGLVLREVGTDVLGSARRVVVSKDDTIIVDGGGSNDAVAKRVNQLRAEIEVSDSEWDREKLQERVAKLAGGVAVIKVGAVTETALKKRKESVEDAVAAAKASIEEGIIAGGGSALVQCGAALKQLRTSLTGDEALGIDVFFEALKAPLYWIATNAGLDGAVVVDKVSGLPAGHGLNASTLGYGDLVADGVVDPVKVTRSAVLNAASVARMMLTTETAVVDKPAKTEEHDHHGHAH

SEQ ID NO: 17 - protein, 541 aa
MAKTIAYDEEARRGLERGLNSLADAVKVTLGPKGRNVVLEKKWGAPTITNDGVSIAKEIELEDPYEKIGAELVKEVAKKTDDVAGDGTTTATVLAQALVKEGLRNVAAGANPLGLKRGIEKAVDKVTETLLKDAKEVETKEQIAATAAISAGDQSIGDLIAEAMDKVGMEGVITVEESNTFGLQLELTEGMRFDKGYISGYFVTDAERQEAVLEEPYILLVSSKVSTVKDLLPLLEKVIQAGKSLLIIAEDVEGEALSTLVVNKIRGTFKSVAVKAPGFGDRRKAMLQDMAILTGAQVISEEVGLTLENTDLSLLGKARKVVMTKDETTIVEGAGDTDAIAGRVAQIRTEIENSDSDYDREKLQERLAKLAGGVAVIKAGAATEVELKERKHRIEDAVRNAKAAVEEGIVAGGGVTLLQAAPALDKLKLTGDEATGANIVKVALEAPLKQIAFNSGMEPGVVAEKVRNLSVGHGLNAATGEYEDLLKAGVADPVKVTRSALQNAASIAGLFLTTEAVVADKPEKTAAPASDPTGGMGGMDF

SEQ ID NO: 18 - protein, 539 aa
MSKLIEYDETARRAMEVGMDKLADTVRVTLGPRGRHVVLAKAFGGPTVTNDGVTVAREIELEDPFEDLGAQLVKSVATKTNDVAGDGTTTATILAQALIKGGLRLVAAGVNPIALGVGIGKAADAVSEALLASATPVSGKTGIAQVATVSSRDEQIGDLVGEAMSKVGHDGVVSVEESSTLGTELEFTEGIGFDKGFLSAYFVTDFDNQQAVLEDALILLHQDKISSLPDLLPLLEKVAGTGKPLLIVAEDVEGEALATLVVNAIRKTLKAVAVKGPYFGDRRKAFLEDLAVVTGGQVVNPDAGMVLREVGLEVLGSARRVVVSKDDTVIVDGGGTAEAVANRAKHLRAEIDKSDSDWDREKLGERLAKLAGGVAVIKVGAATETALKERKESVEDAVAAAKAAVEEGIVPGGGASLIHQARKALTELRASLTGDEVLGVDVFSEALAAPLFWIAANAGLDGSVVVKKVSELPAGHGLNVNTLSYGDLAADGVIDPVKVTRSAVLNASSVARMVLTTETVVVDKPAKAEDHDHHHGHAH

SEQ ID NO: 19 - protein, 545 aa
MAAKDVQFGNEVRQKMVNGVNILANAVRVTLGPKGRNVVVDRAFGGPHITKDGVTVAKEIELKDKFENMGAQMVKEVASKTNDVAGDGTTTATVLAQSIVAEGMKYVTAGMNPTDLKRGIDKAVAALVEELKNIAKPCDTSKEIAQVGSISANSDEQVGAIIAEAMEKVGKEGVITVEDGKSLENELDVVEGMQFDRGYLSPYFINDAEKQIAGLDNPFVLLFDKKISNIRDLLPVLEQVAKASRPLLIIAEDVEGEALATLVVNNIRGILKTVAVKAPGFGDRRKAMLQDIAILTGGTVISEEVGLSLEKATLDDLGQAKRIEIGKENTTIIDGFGDAAQIEARVAEIRQQIETATSDYDKEKLQERVAKLAGGVAVIKVGAATEVEMKEKKDRVEDALHATRAAVEEGVVAGGGVALLRARAALENLHTGNADQDAGVQIVLRAVESPLRQIVANAGGEPSVVVNKVLEGKGNYGYNAGSGEYGDMIEMGVLDPAKVTRSALQHAASIAGLMLTTDCMIAEIPEEKPAMPDMGGMGGMGGMMZ

SEQ ID NO: 20 - protein, 539 aa
MVKQLKFSEDARQAMLRGVDQLANAVKVTIGPKGRNVVLDKEFTAPLITNDGVTIAKEIELEDPYENMGAKLVQEVANKTNEIAGDGTTTATVLAQAMIQEGLKNVTSGANPVGLRQGIDKAVKVAVEALHENSQKVENKNEIAQVGAISAADEEIGRYISEATEKVGNDGVITIITIEESNRLNTELELGMQFDRGYQSPYMVTDSDKMVAELERPYILVTDKKISSFQDILPLLEQVVQSNRPILIVADEVEGDALTNIVLNRMRGTFTAVAVKAPGFGDRRKAMLEDLAILTGAQVITDDLGLDLKDASIDMLGTASKVEVTKDNTTVVDGDGDENSIDARVSQLKSQIEETESDFDREKLQERLAKLAGGVAVIKVGAASETELKERKLRIEDALNSTRAAVEEGIVAGGGTALVNVYQKVSENEAEGDIETGVNIVLKALTAPVRQIAENAGLEGSVIVERLKNAEPGVGFNGATNEWVNMLRRGIVDPTKVTRSALQHAASVAAMFLTTEAVVASIPEKNNDQPNMGGMPGMM

SEQ ID NO: 21 - protein, 541 aa
MAKSIIYNDEARRALERGMDILAEAVAVTLGPKGRNVVLEKKFGSPQIINDGITIAKEIELEDHVENTGVSLIRQAASKTNDVAGDGTTTATVLAHAIVKEGLRNVAAGANPISLKRGIDKATDFLVARIKEHAQPVGDSKAIAQVGAISAGNDEEVGQMIANAMDKVGQEGVISLEEGKSMTTELEITEGMRFDKGYISPYFVTDAERMEAVLEDPRILITDKKINLVQDLVPILEQVARQGKPLLIIAEDIEKEALATLVVNRLRGVLNVAAVKAPGFGDRRKQMLEDIATLTGGQVISEDAGLKLESATVDSLGSARRINITKDNTTIVAEGNEAAVKSRCEQIRRQIEETDSSYDKEKLQERLAKLAGGVAVIKVGAATETEMKDRKLRLEDAINATKAAVEEGIVPGGGTTLAHLAPQLEDWATGNLKDEELTGALIVARALPAPLKRIAENAGQNGAVISERVKEKEFNVGYNAASLEYVDMLAAGIVDPAKVTRSALQNAASIAGMVLTTECIVVDKPEKEKAPAGAPGGDFDY

SEQ ID NO: 22 - protein, 552 aa
MSKLISFKDESRRSLEAGINALADAVRITLGPKGRNVLLEKQYGAPQIVNDGITVAKEIELSNPEENAGAKLIQEVASKTKEIAGDGTTTATIIAQALVREGLRNVAAGANPVALRRGIEKVTTFLVQEIEAVAKPVEGSAIAQVATVSSGNDPEVGAMIADAMDKVTKDGVITVEESKSLNTELEVVEGMQIDRGYISPYFITDSDRQLVEFDNPLILITDKKISAIAELVPVLEAVARAGRPLLIIAEDIEGEALATLVVNKARGVLNVAAIKAPAFGDRRKAVLQDIAILTGGSVISEDIGLSLDTVSLDQLGQAVKATLEKDNTILVAGADKRASAGVKERIEQLRKEYAASDSDYDKEKIQERIAKLAGGVAVIKVGAATETELKDRKLRIEDALNATKAAVEEGIVPGGGTTLIRLAGKIESFKAQLSNDEERVAADIIAKALEAPLHQLASNAGVEGSVIVEKVKEATGNQGYNVITGKIEDLIAAGIIDPAKVVRSALQNAASIAGMVLTTEALVVEKPEPAAPAMPDMGGMGGMGGMGGMGMM

SEQ ID NO: 23 - protein, 539 aa
MAKTIAFDKKARRGLERGLNALADAVKVTLGPKGRNVVLEKKWGAPTITNDGVSIAKEIELEDPYEKIGAELVKEVAKKTDDVAGDGTTTATVLAQALVREGLRNVAAGANPLGLKRGIEKAVEAVTEHLLKAAKEVETKDQIAATAGISAGDPAIGELIAEAMDKVGKEGVITVEESNTFGLQLELTEGMRFDKGFISGYFATDAERQEAVLEDPYVLLVSGKISTVKDLLPLLEKVIQSGKPLAIIAEDVEGEALVTLIVNKIRGTFKSVAIKAPGFGDRRKAMLQDMAILTGGQVISEEIGLSLDTAGLEVLGQARQVVVTKDETTIVDGAGSKEQIAGRVSQIRAEIENSDSDYDREKLQERLAKLAGGVAVIKAGAATEDLKERKHRIEDAVRNAKAAVEEGIVAGGGSSLAQSGTVFDSXALEGDEATGANIVKVALDAPVKQIAVNAGLEPGVVAEKVRNSPAGTGLNAATGVYEDLLAAGINDPVKVTRSALQNAASIAALFLTTEAVVADKPEKAGAPVDPTGGMGGMDF

SEQ ID NO: 24 - protein, 582 aa
MVSFLSSSVSRLPLRIAGRRIPGRFAVPQVRTYAKDLKFGVDARASLLTGVDTLARAVSVTLGPKGRNVLIDQPFGSPKITKDGVTVARSVSLKDKFENLGARLVQDVASKTNEVAGDGTTTATVLTRAIFSETVRNVAAGCNPMDLRRGIQLAVDNVVEFLQANKRDITTSEEISQVATISANGDTHIGELLAKAMERVGKEGVITVKEGRTISDELEVTEGMKFDRGYISPYFITDVKSQKVEFENPLILLSEKKVSAVQDILPSLELAAQQRRPLVIIAEDVDGEALAACILNKLRGQLQVVAIKAPGFGDNRRNMLGDLAVLTDSAVFNDEIDVSIEKAQPHHLGSCGSVTVTKEDTIIMKGAGDHVKVNDRCEQIRGVMADPNLTESEKEKLQERLAKLSGGIAVIKVGASSEVEVNEKKDRIVDALNAVKAAVSEGVLPGAGTSFVKASLRLGDIPTNNFDQKLGVEIVRKAITRPAQTILENAGLEGNLIVGKLKELYGKEFNIGYDIAKDRFVDLNEIGVLDPLKVVRTGLVDASGVASLMGTTECAIVDAPEESKAPAGPPGMGGMGGMPGMM

SEQ ID NO: 25 - protein, 572 aa
MLRSSVVRSRATLRPLLRRAYSSHKELKFGVEGRASLLKGVETLAEAVAATLGPKGRNVLIEQPFGPPKITKDGVTVAKSIVLKDKFENMGAKLLQEVASKTNEAAGDGTTSATVLGRAIFTESVKNVAAGCNPMDLRRGSQVAVEKVIEFLSANKKEITTSEEIAQVATISANGDSHVGKLLASAMEKVGKEGVITIREGRTLEDELEVTEGMRFDRGFISPYFITDPKSSKVEFEKPLLLLSEKKISSIQDILPALEISNQSRRPLLIIAEDVDGEALAACILNKLRGQVKVCAVKAPGFGDNRKNTIGDIAVLTGGTVFTEELDLKPEQCTIENLGSCDSITVTKEDTVILNGSGPKEAIQERIEQIKGSIDITTTNSYEKEKLQERLAKLSGGVAVIRVGGASEVEVGEKKDRYDDALNATRAAVEEGILPGGGTALVKASRVLDEVVVDNFDQKLGVDIIRKAITRPAKQIIENAGEEGSVIIGKLIDEYGDDFAKGYDASKSEYTDMLATGIIDPFKVVRSGLVDASGVASLLATTEVAIVDAPEPPAAAGAGGMPGGMPGMPGMM

SEQ ID NO: 26 - protein, 577 aa
MISTLRGKIFNNGSNRNKCVSILSNIQKRNISKDIRFGSDARTAMLTGCNKLADAVSVTLGPKGRNVIIEQSFGSPKITKDGVTVAKSIEFNNKLANLGAQMVKQVAANTNGKAGDGTTTATILARSIFQQGCKAVDSGMNPMDLLRGINKGVEKVLEYLNSIKKDVTTTEEIFNVASISNGDKNIGQLIADTMKKVGKEGTITVTEGKTLQHELEIVEGIKFDRGYISPYFINNSQKVELDKPYILIHEKKISTVKSLLPVLEHVLQNQSSLLVIAEDVDSDALATLIVNKLRLGLKICAVKAPGFGEHRKALIHDIAVMTGAKVITEETGLKLDDPQVVSYLGKAKSINVTKDSTLIMEGEGKKEEINERCESIRNAIKMNTSDYEKEKLQERLAKITGGVALIKVGGISEVEVNEIKDRIQDALCATKAAVEEGIVPGGGSALLFASKELDSVQTDNYDQRVGVNIIKDACKAPIKQIAENAGHEGSVVAGNILKDKNSNIGFNAQEGKYVDMIESGIIDPTKVVKTAISDAASIASLMTTTEVAIVDFKDSKNEESSQHMNSVNSMGDMGGMY

SEQ ID NO: 27 - protein, 550 aa
MTNVVVSGEQLQQAFREVAAVIDSTVAVTAGPRGKTVGINKPYGAPEITKDGYKVMKGIKPEKPLNAAITSIFAQSCSQCNDKVGDGTTTCSILTSGMIVEASKSIAAGNDRISIKNGMQKAKDVVLKEVASMARTISLEKIDEVAQVAIISANGDRSIGSNIADAVKKVGKEGVITVEESKGSKELEVELTTGMQFDRGYLSPYFITNNEKMIVELDDPYLLITEKKLNIIQPLLSILEAVVKSGKPLLIIAEDIEGEALSTLVINKLRGGLKVAAVKAPGFGDRRKEMLEDIAALTNAKYVIKDELGIKMEDLTLEDLGIAKNVKITKDNTTIVSENRVTDRVKARIEQIKSQIESSTSDYDKEKLRERLAKLSGGVAVLKVGGATELEVKERRDRVEDQLHATRAAIEEGIVPGGGVALLYASSALDKLKGADDEEQIGINIIKKVLSVPIKRLVKNAGLESAVIIDYLIKQNNKELIYNVEAMSYANAFAAGVIDPAKVVRIAFETAISVASVLITTESMIVDIPNKDENASSPMGAGGMGRMNDF

SEQ ID NO: 28 - protein, 568 aa
MLRLARKGLQTAVVRSYAKDVKFGAEGRQAMLVGVNLLADAVSVTMGPKGRNVIIEQSWGSPKITKDGVTVAKSIDLKDKYQNLGAKLIQDVANKANEEAGDGTTCATVLTRAIAKEGFERHSSRGNAVEIRRGVMNAVEVVVAELKKISKKVTTPEEIAQVATISANGDTVVGNLISDAMKKVGTTGVITVKDGKTLNDQLELIEGMKFDRGYISPYFITSAKGAKVEYEKALVLLSEKKISQVQDIVPALELANKLRRPLVIIAEDVDGEALTTLVLNRLKVGLQVVAIKAPGFGDNRKNALKDMGIATGASIFGDETLDLRLEDITANDLGEVDEVTITKDDTLLLRGRGDQTEIEKRIEEITDEIERSTSDYEKEKLNERLAKLSKGVAVLKIGGGSEVEVGEKKDRVTDALCATRAAVEEGIVPGGGVALLRSLTALKNYKAANEDQQIGVNIVKKALTQPIATIVKNAGLEPSSIIDEVTGNSNTSYGYDALNGKFVDMFEAGIIDPTKVVRTALQDASGVASLLATTECVVTEIPKEEAVGGPAGGMGGMGGMGGMGGMGF

SEQ ID NO: 29 - protein, 576 aa
MFRLPVSLARSSISRQLAMRGYAKDVRFGPEVRAMMLQGVDVLADAVAVTMGPKGRNVIIEQSVGLAKITKDGVTVAKSIELKDKFQNIGAKLVQDLANNTNEEAGDGTTTATFLARAIAKEGFEKISKGGNPVEIRRGVMLAVETVKDNLKTMSRPVSTPEEIAQVATISANGDREIGNGKVSVSEAMKKVGRDGVITVKDGKTLTDELEVIEGTMRFDRGYISPYFINSSKGAKVEFQDALLLLSEKKISSVAEHHSPLWRLASRRTRKPLVIIAEDIDGEALSTLVVNRLKIGLQVAAVKAPGFGDNRKSTLTDMATSGGIVFGDDVSLVKLEDVKVSDLGQVGEVVITKDDTLLLKGKGKKDDVLRRANQIRTKIEDTTSEYEKEKLQERLARLASGVALRVGGSSEVEVNEKKDRVHDALNATRAAVEEGIVPGGGRPLLRCIEKLEGVETTNEDQKLGVEIVRRALRMPCMTIAKNAGVDGAMVVAKVENQAGDYGYDAKGEYGNLIEKGIIDPTKVVRTAITDASGVASLLTTAEAVVTEIPKEDGAPAMPGMGGMGGMGGMGGMGGMM

SEQ ID NO: 30 - protein, 573 aa
MLRLPTVFRQMRPVSRVLAPHLTRAYAKDVKFGADARALMLQGVDLLADAVAVTMGPKGRTVIIEQGWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 31 - protein, 577 aa
MYRFASNLASKARIAQNARQVSSRMSWSRNYAAKEIKFGVEARALMLKGVEDLADAVKVTMGPKGRNVVIEQSWGAPKVTKDGVTVAKSIEFKDKIKNVGASLVKQVANATNDVAGDGTTCATVLTRAIFAEGCKSVAAGMNAMDLRRGISMAVDAVVTNLKSKARMISTSEEIAQVGTISANGEREIGELIAKAMEKVGKEGVITIQDGKTLFNELEVVEGMKLDRGYTSPYFITNQKTQKCELDDPLILIHEKKISSINSIVKVLELALKRQRPLLIVSEDVESDALATLILNKLRAGIKVCAIKAPGFGENRKANLQDLAALTGGEVITDELGMNLEKVDLSMLGTCKKVTVSKDDTVILDGAGDKKGIEERCEQIRSAIELSTSDYDKEKLQERLAKLSGGVAVLKIGGASEAEVGEKKDRVTDALNATKAAVEEGILPGGGVALLYAARELEKLPTANFDQKIGVQIIQNALKTPVYTIASNAGVEGAVIVGKLLEQDNPDLGYDAAKGEYVDMVKAGIIDPLKVIRTALVDAASVSSLLTTTEAVVVDLPKDESESGAAGGGMGGMVVMDY

SEQ ID NO: 32 - protein, 576 aa
MYRAAASLASKARQAGSSSAARQVGSRLAWSRNYAAKDIKFGVEARALMLRGVEELADAVKVTMGPKGRNVVIEQSFGAPKVTKDGVTVAKSIEFKDRVKNVGASLVKQVANATNDNAGDGTTCATVLTKAIFTEGCKSVAAGMNAMDLRRGISMAVDAVVTNLKGMARMISTSEEIAQVGTISANGEREIGELIAKAMEKVGKEGVITIADGNTLYNELEVVEGMKLDRGYISPYFITNSKAQKCEPEDPLILIHDRKVTNMHAVVKVLEMALKKQRPLLIVAEDVESEALGTLIINKLRAGIKVCAVKAPGFGENRKANLQDLAILTGGEVITEELGMNLENVEPHMLGSCKKVTVSKDDTVILDGAGDKKSIEERADQIRSAVENSTSDYDKEKLQERLAKLSGGVAVLKIGGASEAEVGEKKDRVTDALNATKAAVEEGIVPGGGVALLYASKELDKLQTANFDQKIGVQIIQNALKTPVHTIASNAGVEGAVVVGKLLEQGNTDLGYDAAKDEYVDMVKAGIIDPLKVIRTALVDAASVSSLMTTTESIIVEIPKEEAPAPAMGGMGGMDY

SEQ ID NO: 33 - protein, 587 aa
MASTNALSSTSILRSPTNQAQTSLSKKVKQHGRVNFRQKPNRFVVKAAAKDIAFDQHSRSAMQAGIDKLADAVGLTLGPRGRNVVLDEFGSPKVVNDGVTIARAIELPDPMENAGAALIREVASKTNDSAGDGTTTASILAREIIKLGLLNVTSGANPVSIKKGIDKTVAALVEELEKLARPVKGGDDIKAVATISAGNDELIGKMIAEAIDKVGPDGVLSIESSNSFETTVEVEEGMEIDRGYISPQFVTNPEKSIVEFENARVLITDQKISAIKDIIPLLEKTTQLRAPLLIISEDITGEALATLVVNKLRGILNVAAIKAPGFGERRKALLQDIAILTGAEFQASDLGLLVENTTIEQLGLARKVTISKDSTTIIADAASKDELQSRVAQLKKELSETDSIYDSEKLAERIAKLSGGVAVIKVGAATETELEDRKLRIEDAKNATFAAIEEGIVPGGGTALVHLSGYVPAIKEKLEDADERLGADIVQKALVAPAALIAQNAGIEGEVVVEKIKNGEWEVGYNAMTDTYENLVESGVIDPAKVTRCALQNAASVAGMVLTTQAIVVEKPKPKAAVAAAPQGLTI

SEQ ID NO: 34 - protein, 545 aa
MAKDIKFGEEARRAMLRGVNALADAVKVTLGPKGRNVVLEKSFGAPTITKDGVTVAKEIELEDKFENMGAQLVKEVASKTNDVAGDGTTTATVLAQAIVKEGLKNVAAGANPMDLRRGIDKAVDAVVEELKAIAKPVETKEEIAQVATISANGDEEIGELIAEAMEKVGKEGVITVEEGKTLETELEVVEGMQFDRGYISPYFITDSEKQKAELEDPLILLTDKKISNIQDLLPVLEEVAQAGKPLLIIAEDVEGEALATLVVNKLRGTLKVVAVKAPGFGDRRKAMLQDIAILTGGQVISEELGLSLEDATLEDLGQAKKVVVTKDDTTIVDGAGDAAIAGRVAQIRSQIEESTSDYDKEKLQERLAKLAGGVAVIKVGAATEVELKERKDRVEDALNATRAAVEEGIVPGGGVALLRAAPALDKLKTENGDEATGVNIVLRALEAPLRQIAENAGLEGSVVVEKVKNSEAGGYNAATGEYVDMIAAGIIDPTKVTRSALQNAASVASLMLTTEAVVVDKPEKEAAPAGMPGMMGGMGGMGGMM

SEQ ID NO: 35 - DNA, 24 bp; single-stranded, linear
catatggcngcnaaagaygtaaaa

SEQ ID NO: 36 - DNA, 24 bp; single-stranded, linear
tgatcacatcatnccncccatncc

SEQ ID NO: 37 - DNA, 24 bp; single-stranded, linear
catatggcaaaagaaathaartty

SEQ ID NO: 38 - DNA, 24 bp; single-stranded, linear
tgatcanccncccatnccncccat

SEQ ID NO: 39 - DNA, 16 bp; single-stranded, linear
gtaaaacgacggccag

SEQ ID NO: 40 - DNA, 17 bp; single-stranded, linear
caggaaacagctatgac

SEQ ID NO: 41 - DNA, 17 bp; single-stranded, linear
ccaaccatcacgaaaga

SEQ ID NO: 42 - DNA, 17 bp; single-stranded, linear
acgggtcactttggttg

SEQ ID NO: 43 - DNA, 17 bp; single-stranded, linear
ttactaatgacggggta

SEQ ID NO: 44 - DNA, 17 bp; single-stranded, linear
ttaccaatgacggtgtg

SEQ ID NO: 45 - DNA, 17 bp; single-stranded, linear
acagggtcaatgattcc

SEQ ID NO: 46 - DNA, 17 bp; single-stranded, linear
actggatcaatgatacc

SEQ ID NO: 47 - DNA, 17 bp; single-stranded, linear
ccgtaccgtgctctgac

SEQ ID NO: 48 - DNA, 17 bp; single-stranded, linear
accacgtttcagatcca

SEQ ID NO: 49 - DNA, 17 bp; single-stranded, linear
gacagtttcgcggcaac

SEQ ID NO: 50 - DNA, 17 bp; single-stranded, linear
ctcagaacgaagatcag

SEQ ID NO: 51 - DNA, 17 bp; single-stranded, linear
ggtatgcagttcgaccg

SEQ ID NO: 52 - DNA, 17 bp; single-stranded, linear
ccgtgttggtcaaatcc

SEQ ID NO: 53 - DNA, 17 bp; single-stranded, linear
ggtaactacggttacaa

SEQ ID NO: 54 - DNA, 17 bp; single-stranded, linear
gaggccacttctttcac

SEQ ID NO: 55 - DNA, 17 bp; single-stranded, linear
ggcttccagcactggca

SEQ ID NO: 56 - DNA, 17 bp; single-stranded, linear
aacttcagtcgcagcac

SEQ ID NO: 57 - DNA, 17 bp; single-stranded, linear
ccttgaaagccattgct

SEQ ID NO: 58 - DNA, 17 bp; single-stranded, linear
gctacacgtgcagccgt

SEQ ID NO: 59 - DNA, 17 bp; single-stranded, linear
gctgcaacaggtgagtg

SEQ ID NO: 60 - DNA, 17 bp; single-stranded, linear
tcatgaacaatggcttg

SEQ ID NO: 61 - DNA, 17 bp; single-stranded, linear
acgaagcacaatgttac

SEQ ID NO: 62 - DNA, 17 bp; single-stranded, linear
atcactaaagatggtgt

SEQ ID NO: 63 - DNA, 17 bp; single-stranded, linear
gcagttgccgcagcagt

SEQ ID NO: 64 - DNA, 17 bp; single-stranded, linear
gctactcgtgcagctgt

SEQ ID NO: 65 - DNA, 17 bp; single-stranded, linear
gttctccgtgctttgga

SEQ ID NO: 66 - DNA, 17 bp; single-stranded, linear
gcacctgctgtgacgtt

SEQ ID NO: 67 - DNA, 17 bp; single-stranded, linear
tcttcgatggtgatgac

SEQ ID NO: 68 - DNA, 17 bp; single-stranded, linear
ggcaagagctgttccgc

SEQ ID NO: 69 - DNA, 17 bp; single-stranded, linear
ctgagccagtacggttg

SEQ ID NO: 70 - DNA, 17 bp; single-stranded, linear
gtactgcagagcggaac

SEQ ID NO: 71 - DNA, 17 bp; single-stranded, linear
accgtcttcaacggtga

SEQ ID NO: 72 - DNA, 17 bp; single-stranded, linear
gttatcattgctgaaga

SEQ ID NO: 73 - DNA, 17 bp; single-stranded, linear
acggtaccgccggtcag

SEQ ID NO: 74 - DNA, 17 bp; single-stranded, linear
ctgggccaggctaaacg

SEQ ID NO: 75 - DNA, 17 bp; single-stranded, linear
cgactgaagttgaaatg

SEQ ID NO: 76 - DNA, 17 bp; single-stranded, linear
gctgttgaagaactgaa

SEQ ID NO: 77 - DNA, 17 bp; single-stranded, linear
gtcttcaacggtgatca

SEQ ID NO: 78 - DNA, 17 bp; single-stranded, linear
tcttctaccgcagcacg

SEQ ID NO: 79 - DNA, 17 bp; single-stranded, linear
ctcttgattattgcgga

SEQ ID NO: 80 - DNA, 17 bp; single-stranded, linear
ttgttcaaaacaagagt

SEQ ID NO: 81 - DNA, 17 bp; single-stranded, linear
cgattattgtagaaggt

SEQ ID NO: 82 - DNA, 17 bp; single-stranded, linear
cttgataaccgcaacac

SEQ ID NO: 83 - DNA, 17 bp; single-stranded, linear
tccaaagcacggagaac

SEQ ID NO: 84 - DNA, 17 bp; single-stranded, linear
gtgtcaaacatccaaga

SEQ ID NO: 85 - DNA, 17 bp; single-stranded, linear
tcttcgatggtaatcac

SEQ ID NO: 86 - DNA, 17 bp; single-stranded, linear
gcaataatgagtaatgg

SEQ ID NO: 87 - DNA, 17 bp; single-stranded, linear
acagtaattgttgaagg

SEQ ID NO: 88 - DNA, 17 bp; single-stranded, linear
cagtgcaatacggttag

SEQ ID NO: 89 - DNA, 17 bp; single-stranded, linear
agcttccagaaccggca

SEQ ID NO: 90 - DNA, 17 bp; single-stranded, linear
ctgatcatcgctgaaga

SEQ ID NO: 91 - DNA, 16 bp; single-stranded, linear
acggttattgtagaag

(19) United States
(12) Patent Application Publication
(10) Pub. No.: US 2006/0039922 A1
(43) Pub. Date: Feb. 23, 2006

(54) Streptococcal heat shock proteins of the Hsp60 family

(75) Inventors: Lee Mizzen, Victoria (CA); Jan Wisniewski, Sooke (CA)

Correspondence Address: FISH & RICHARDSON PC, P.O. BOX 1022, MINNEAPOLIS, MN, 55440-1022, US

(73) Assignee: Stressgen Biotechnologies Corporation, a Canadian corporation

(21) Appl. No.: 11/201,916

(22) Filed: Aug. 11, 2005

Related U.S. Application Data
(63) Continuation of application No. 09/001,737, filed on Dec. 31, 1997, now abandoned.

(51) Int. Cl. A61K 39/02; C07H 21/04; C07K 14/315; C12N 15/74

(52) U.S. Cl. 424/190.1; 435/69.3; 435/320.1; 435/252.3; 530/350; 536/23.7

(57) ABSTRACT

Methods and compositions comprising isolated nucleic acid molecules specific to Streptococcus pneumoniae and Streptococcus pyogenes, as well as vector constructs and isolated polypeptides specific to Streptococcus pneumoniae and Streptococcus pyogenes are provided. Such compositions and methods are useful for the diagnosis of Streptococcal infection and for generating an immune response to Streptococcal bacteria.

### CROSS-REFERENCE TO RELATED APPLICATIONS

The present application is a continuation (and claims the benefit of priority under 35 USC 120) of U.S. application Ser. No. 09/001,737, filed Dec. 31, 1997, the contents of which are herein incorporated by reference.

### TECHNICAL FIELD OF THE INVENTION

This invention relates to Streptococcal Hsp60 proteins, including fragments thereof, and nucleic acid molecules encoding such proteins and fragments, in particular from Streptococcus pneumoniae and Streptococcus pyogenes, and uses of such proteins and nucleic acid molecules.

### BACKGROUND OF THE INVENTION

The World Health Organization has estimated that, worldwide, about 30% of deaths of children under age 5, or about 4-5 million, result from acute respiratory infections. David Klein, Pneumococcal Conjugate Vaccines: Review and Update, in Microbial Drug Resistance 1:49, 1995. The most frequent causative agent responsible for these deaths is Streptococcus pneumoniae, which is also referred to as pneumococcus and causes a wide variety of infections such as sinusitis, otitis, pneumonia, bacteremia and meningitis. This organism is found on respiratory mucosal membranes of 15-35% of healthy children and up to 80% of children with respiratory infections. Gray et al., J. Infect. Dis. 142:923, 1980; Hendley et al., J. Infect. Dis. 132:55, 1975. In addition, Streptococcus pneumoniae is responsible for 70,000 meningitis deaths and a similar number of deaths from sepsis and other infections.

In developing countries, Pneumococcal infections are responsible for approximately 1.2 million deaths among children 5 years of age and younger, which corresponds to nearly 40% of all pneumonia related deaths. World Health Organization, Pneumococcal Conjugate Vaccines, reported in Report of Meeting of Nov. 15-17, 1993 (WHO/ARI/94.34). In the industrialized world, taking the U.S. as an example, pneumococcus is a leading cause of severe morbidity in the general population and of death in the elderly as well as the immunocompromised population. Klein et al. (supra). Pneumococcus causes more deaths (about 50,000) in older adults than any other infectious agent. High risk individuals include those with sickle cell anemia, nephrotic syndrome, asplenia, alcoholism and HIV infection. Pneumococcus also poses a large risk to children under the age of two. In infants below the age of two, pneumococcus is the predominant cause of meningitis, bacteremia and otitis media. Within the first two years of life, about 25% of children experience otitis media caused by pneumococcus, a percentage that increases to 75% by the age of six. In Finland, two-year old children have experienced, on average, more than one episode of otitis media. About half of the cases of acute otitis media were determined to be caused by pneumococcus. Eskola, J. and Kaeyhty, H., Ann. Med. 27:53, 1995.

Pneumococcus is a gram-positive organism that has type-specific capsular polysaccharides. Eighty-three different type specificities have been identified and have been designated 1-83 in the American system. Jennings, Current Topics in Microbiology and Immunology 150:97-121, 1990. The structures of the different Pneumococcal polysaccharides have been reviewed by Kenne and Lindberg in The Polysaccharides 2:282-363 (Aspinal ed., 1983).

The need for an effective way to generate an immune response against Streptococcus pneumoniae was recognized long ago. In 1945 it was demonstrated that isolated capsular polysaccharides were able to provide type-specific protection in humans. MacLeod et al., J. Exp. Med. 82:445-65, 1945. However, this protection was inadequate due to the large number of different polysaccharides needed for complete protection. The interest in a vaccine soon subsided due to the success of antibiotic treatment of infections.

Recently the interest in the development of an effective vaccine has renewed. One reason was that antibiotic treatment of infectious diseases caused by encapsulated bacteria such as pneumococcus did not always prevent morbidity and mortality. For an analogous example, cured Hemophilus influenzae meningitis was a major cause of acquired mental retardation. Sell et al., Pediatrics 49:206-11, 1972. Another important reason for the renewed interest in vaccine development was the appearance and rapid spread of antibiotic-resistant strains of pneumococcus. For example, in two hospitals in Paris, France, the frequency of resistant isolates from patients increased from 1.8% in 1987 to 17% in 1990. In Barcelona, Spain, the rate of resistance increased from 4.3% in 1979 to 40% in 1990. See Lonks and Medeiros, Antimicrobial Therapy 1 79:523-35, 1995. Multidrug-resistant pneumococcus have also appeared in many countries including 18 of the 50 states of the United States.

A vaccine containing polysaccharide antigens for 14 of the 83 capsular types was developed and released in 1978. Lonks and Medeiros, supra. This vaccine, was improved in 1983 by the creation of second generation vaccine containing 23 different polysaccharides. However, two large studies, using this vaccine, one with 2837 patients, showed that the improved vaccine was only about 57% efficacious against Pneumococcal bacteremia. Butler et al., JAMA 270:1826, 1993.

A drawback to polysaccharide-based vaccines is that the efficacy of these vaccines is problematic in infants under two years of age, who respond very poorly to these vaccines. Gotschlich et al., Antibodies in Human Diagnosis and Therapy 391-402 (Haber and Krause eds., 1977). An additional drawback is that antibodies produced by polysaccharide-based vaccines are predominantly of the IgM isotype, and therefore the immune response is not heightened upon secondary exposure to the antigen.

These and other concerns about polysaccharide-based vaccines demonstrate that there is a need in the art for improved compositions which can be used to generate an immunogenic response directed to Streptococcus pneumoniae.

Turning to Streptococcus pyogenes, also referred to as group A streptococcus (“GAS”), it too is a gram-positive bacterium that is causatively associated with a number of human disease states, ranging from acute pharyngitis (strep throat) to invasive diseases involving degeneration of the heart valves (acute rheumatic fever) and acute post-Streptococcal glomerulonephritis. Facklam, Development of Group A Streptococcal Vaccines, in Manual of Clinical Microbiology 1-22 (Lennette, Balows, Hausler and Truant eds., 1980). Infection by this bacterium can also cause impetigo (a supporative mucosal infection), invasive fasciitis (viz. flesh-eating disease), boils and skin abscesses (pyoderma), scarlet fever, sepsis, a severe toxic-shock like syndrome and pneumonia.

Before the advent of antibiotic therapy, rheumatic fever was a leading cause of mortality in children and of chronic heart disease in individuals who survived systemic infection. In developing countries, rheumatic fever is still an enormous problem. It has been estimated that in India over 6 million school age children suffer from rheumatic heart disease. Agarwal, Lancet I 910-11, 1981. In the United States, the CDC has estimated that 25-40 million cases of Streptococcus pyogenes-induced pharyngitis occur every year, costing over $2 billion for physician visits, culture work and antibiotic therapy. There also has been an increase in toxic-shock like syndrome caused by the organism. Presently, 10,000-15,000 cases of Streptococcal and Staphylococcal Toxic shock like infections occur annually in the United States. While presently GAS infections are treated with antibiotics, given what is known about other bacteria including pneumococcus (as detailed above), the proliferation of antibiotic-resistant strains is a concern.

GAS are differentiated from other streptococci by their Group A carbohydrate, a cell wall moiety containing rhamnose and N-acetyl glucosamine. Different strains of GAS are classified, serologically, based on their M protein or on the T antigen. GAS can be assigned to 80-100 different M protein groups which form the principal basis for characterizing pathological strains. The M protein is a surface protein and is both a major virulence factor and a major protective antigen. Lancefield, J. Immunol. 89:307, 1962. Antibodies against M protein are opsonic and promote killing of the bacteria by phagocytes. Lancefield, supra.

While M proteins are potentially useful in the constitution of a vaccine, several obstacles remain on the route to an effective vaccine. First, the M protein contains epitopes that cross-react with human tissue, especially the myocardium. Dale and Beachy, J. Exp. Med. 161:113, 1985. Thus, anti-M protein antibodies may cause disease rather than preventing it. Second, it may not be practical to produce a vaccine against all 80-100 different strains of GAS. Any vaccine containing only a few types of M protein may be only partially effective. While the first problem might be overcome by using M protein fragments that lack the cross-reactive epitopes as immunogens (Dale et al., J. Immunol. 151:2188-94, 1993), such an approach has not yet been proven, and the latter problem of immunizing against numerous distinct M proteins still needs to be overcome. Accordingly, there is a need in the art for a composition which provides generates an immunogenic response to S. pyogenes that is not based on the antigenicity of the M proteins.

### SUMMARY OF THE INVENTION

The present invention provides methods and compositions comprising isolated nucleic acid molecules specific to Streptococcus pneumoniae and Streptococcus pyogenes, as well as vector constructs and isolated polypeptides specific to Streptococcus pneumoniae and Streptococcus pyogenes. Such compositions and methods are useful for the diagnosis of Streptococcal infection and for generating an immune response to Streptococcal bacteria.

Thus, in one aspect the present invention provides an isolated nucleic acid molecule encoding a Streptococcus pneumoniae Hsp60 and/or a Streptococcus pyogenes Hsp60. In some embodiments, the isolated nucleotide molecule is selected from the group consisting of: (a) an isolated nucleic acid molecule comprising the sequence of SEQ ID NO:1 from nucleotides 15-1652; (b) an isolated nucleic acid molecule comprising the sequence of SEQ ID NO:3 from nucleotides 15-1640; (c) an isolated nucleic acid molecule comprising the sequence of SEQ ID NO:5 from nucleotides 15-1649; (d) an isolated nucleic acid molecule comprising the sequence of SEQ ID NO:7 from nucleotides 15-1652; (e) an isolated nucleic acid molecule complementary to any one of the nucleotides of SEQ ID NOS: 1, 3, 5 or 7 set forth in (a) through (d), respectively; (f) an isolated nucleic acid molecule that hybridizes under conditions of high stringency to the nucleic acid molecules of any one of (a) through (e).

In another aspect in one aspect the present invention provides an isolated nucleic acid molecule that specifically hybridizes to the nucleic acid molecule of any one of SEQ ID NO:1 from nucleotides 15-1652, SEQ ID NO:3 from nucleotides 15-1640, SEQ ID NO:5 from nucleotides 15-1649, or SEQ ID NO:7 from nucleotides 15-1652 or a complement thereof under conditions of high stringency. In further aspects the present invention provides an isolated nucleic acid molecule comprising a nucleotide sequence that is identical to a segment comprising at least 25% of contiguous nucleotide bases of any one of SEQ ID NO:1 from nucleotides 15-1652, SEQ ID NO:3 from nucleotides 15-1640, SEQ ID NO:5 from nucleotides 15-1649, or SEQ ID NO:7 from nucleotides 15-1652 or a complement thereof or an isolated nucleic acid molecule encoding Hsp60 comprising a nucleic acid sequence that encodes a polypeptide comprising any one of SEQ ID NOS: 2, 4, 6 or 8 or a variant Hsp60 that is at least 95% homologous to a polypeptide according to any one of SEQ ID NOS: 2, 4, 6 or 8.

In one embodiment, the present invention provides an isolated nucleic acid molecule according as described above, the molecule encoding a polypeptide that is able to be selectively bound by an antibody specific for a Streptococcus pneumoniae Hsp60 or a Streptococcus pyogenes Hsp60.

In still another aspect in one aspect the present invention provides an isolated nucleic acid molecule encoding at least 8 amino acids of a Streptococcal Hsp60 polypeptide selected from amino acid residues 1-545 of SEQ ID NO:2, amino acid residues 1-541 of SEQ ID NO:4, amino acid residues 1-544 of SEQ ID NO:6, and amino acid residues 1-545 of SEQ ID NO:8, wherein the encoded Streptococcal Hsp60 polypeptide is able to bind to a major histocompatibility complex.

In still further aspects the present invention provides an isolated Streptococcus pneumoniae Hsp60 polypeptide and an isolated Streptococcus pyogenes Hsp60 polypeptide.

In some embodiments, the isolated Hsp60 polypeptide comprises the amino acid sequence of any one of a Streptococcal Hsp60 polypeptide selected from amino acid residues 1-545 of SEQ ID NO:2, amino acid residues 1-541 of SEQ ID NO:4, amino acid residues 1-544 of SEQ ID NO:6, and amino acid residues 1-545 of SEQ ID NO:8, or variants thereof, preferably wherein the polypeptide is able to be selectively bound by an antibody specific for either a Streptococcus pneumoniae Hsp60 and/or Streptococcus pyogenes Hsp60. In further embodiments, the isolated Hsp60 polypeptide is fused to an additional polypeptide to create a fusion protein.

In still yet further aspects the present invention provides an isolated Hsp60 polypeptide comprising at least 8 amino acids selected from amino acid residues 1-545 of SEQ ID NO:2, amino acid residues 1-541 of SEQ ID NO:4, amino acid residues 1-544 of SEQ ID NO:6, and amino acid residues 1-545 of SEQ ID NO:8, wherein the Hsp60 polypeptide is capable of binding to a major histocompatibility complex and eliciting or enhancing an immune response to Streptococcus in a human being.

In certain embodiments, the isolated Hsp60 polypeptide is derived from proteolytic cleavage or chemical synthesis, is an expression product of a transformed host cell containing a nucleic acid molecule encoding the Hsp60 or portion thereof. In further certain embodiments, the isolated Hsp60 polypeptide comprises greater than 95% homology to any one of a Streptococcal Hsp60 polypeptide selected from amino acid residues 1-545 of SEQ ID NO:2, amino acid residues 1-5410 of SEQ ID NO:4, amino acid residues 1-544 of SEQ ID NO:6, and amino acid residues 1-545 of SEQ ID NO:8, and wherein the Hsp60 polypeptide is able to be selectively bound by an antibody specific for either a Streptococcus pneumoniae Hsp60 or Streptococcus pyogenes Hsp60 or both.

In still yet another aspect the present invention provides an isolated polypeptide wherein the polypeptide is an expression product of a transformed host cell containing one or more of the nucleic acid molecules described herein.

In still yet further aspects the present invention provides vectors comprising one or more of the nucleic acid molecules described herein. In certain embodiments, the vector is an expression vector comprising a promoter in operative linkage with the isolated nucleic acid molecule encoding the Hsp60 or portion thereof, preferably further comprising a selectable or identifiable marker and/or wherein the promoter is a constitutive or an inducible promoter. The present invention also provides host cells containing such vectors. In certain embodiments, the host cell is selected from the group consisting of a bacterial cell, a mammalian cell, a yeast cell and an insect cell.

In still yet other aspects the present invention provides compositions comprising an Hsp60 polypeptide as described herein in combination with a pharmaceutically acceptable carrier or diluent. In certain embodiments, the composition is suitable for systemic administration, oral administration or parenteral administration.

In yet other aspects the present invention provides methods for eliciting or enhancing an immune response in a mammal against Streptococcus, comprising administering to the mammal an effective amount of an Hsp60 polypeptide as described herein in combination with a pharmaceutically acceptable carrier or diluent, methods for eliciting or enhancing an immune response in a mammal against a target antigen comprising administering to the mammal the target antigen joined to an Hsp60 polypeptide as described herein in combination with a pharmaceutically acceptable carrier or diluent.

In another aspect the present invention provides compositions comprising an isolated nucleic acid molecule as described herein wherein the isolated nucleic acid molecule encodes a polypeptide having at least one amino acid difference from a corresponding polypeptide of an Hsp60 protein from an organism other than Streptococcus.

These and other aspects of the present invention will become evident upon reference to the present specification and the attached drawings. In addition, various references are set forth herein that describe in more detail certain procedures or compositions (e.g., plasmids, etc.); all such references are incorporated herein by reference in their entirety.

### BRIEF DESCRIPTION OF THE FIGURES

FIG. 1 depicts the nucleotide and amino acid sequences of Streptococcus pneumoniae Hsp60-1 gene (SEQ ID NOS: 1 and 2 respectively).

FIG. 2 depicts the nucleotide and amino acid sequences of Streptococcus pneumoniae Hsp60-2 gene (SEQ ID NOS: 3 and 4 respectively).

FIG. 3 depicts the nucleotide and amino acid sequences of Streptococcus pyogenes Hsp60-l gene (SEQ ID NOS: 5 and 6 respectively).

FIG. 4 depicts the nucleotide and amino acid sequences of Streptococcus pyogenes Hsp60-2 gene (SEQ ID NOS: 7 and 8 respectively).

FIG. 5 is a schematic representation of the sequencing strategy used to deduce the sequences of the Hsp60 genes from S. pneumoniae and S. pyogenes.

FIGS. 6-9 depict maps of expression vectors pETP60-1, pETP60-2, pETY60-1, and pETY60-2, which vectors include the Hsp60 genes from S. pneumoniae or S. pyogenes, respectively.

FIG. 10 depicts a comparison of the S. pneumoniae (SEQ ID NOS: 2 and 4) and S. pyogenes (SEQ ID NOS: 6 and 8) Hsp60 genes with similar genes from other organisms (SEQ ID NOS: 9 through 34).

FIG. 11 depicts RP-HPLC chromatograms of the Hsp60 genes from S. pneumoniae and S. pyogenes.

### DETAILED DESCRIPTION OF INVENTION

The present invention provides methods and compositions comprising isolated nucleic acid molecules and polypeptides specific to Streptococcus pneumoniae and Streptococcus pyogenes, as well as vector constructs, antibodies and other materials related to isolated nucleic acid molecules and polypeptides. Such compositions and methods are useful for the diagnosis of Streptococcal infection and for generating an immune response to Streptococcal bacteria.

A “stress gene,” also known as “heat shock gene,” is a gene that is activated or otherwise detectably upregulated due to the contact or exposure of an organism (containing the gene) to a stressor, such as heat shock or glucose deprivation or glucose addition. A given “stress gene” also includes homologous genes within known stress gene families, such as certain genes within the Hsp60, Hsp70 and Hsp90 stress gene families, even though such homologous genes are not themselves induced by a stressor. As defined herein, a “stress protein,” also known as a “heat shock protein,” (“Hsp”) is a protein that is encoded by a stress gene, and is therefore typically produced in significantly greater amounts upon the contact or exposure to the stressor of the organism. Each of the terms stress gene and stress protein as used in the present specification are inclusive of the other, unless the context indicates otherwise. Streptococcal Hsps, as well as Hsps from other organisms, appear to participate in important cellular processes such as protein synthesis and assembly and disassembly of protein complexes.

A variety of stress genes and proteins are well known in the art and include, for example, Hsp100-200, Hsp100, Hsp90, Lon, Hsp70, Hsp60, TF55, Hsp40, FKBPs, cyclophilins, Hsp20-30, ClpP, GrpE, Hsp10, ubiquitin, calnexin, peptidyl-prolyl cis-trans isomerases, and protein disulfide isomerases. Macario, A. J. L., Int. J. Clin. Lab. Res. 25:59-70, 1995; Parsell, D. A., & Lindquist, S., Ann. Rev. Genet. 27: 437-496 (1993); U.S. Pat. No. 5,232,833 (Sanders et al.).

In bacteria, the predominant stress proteins are proteins with molecular sizes of about 60 and 70 kDa (i.e., Hsp60 and Hsp70, respectively). Hsp70 and Hsp60 typically represent about 1-3% of bacterial cell protein based on the staining pattern using sodium dodecyl sulfate-polyacrylamide gel electrophoresis (“SDS-PAGE”) and the stain coomassie blue, but accumulate to levels as high as 25% under stressful conditions. Thus, Hsps are produced in an invading bacterium due to stresses put on the bacterium by the environment of the animal, and the Hsps become some of the most significant bacterial antigens displayed to the host and to which the host mounts an immune response. Therefore, by administering a Streptococcal Hsp to an animal, the Streptococcal Hsp can induce an immune response in the animal to Streptococcus, preferably providing resistance to such a bacterial infection. Accordingly, the isolation of Streptococcal Hsp60 genes provides a platform for the generation of isolated polypeptides or fragments or variants of Streptococcal Hsp60 useful in diagnosis and inhibition of Streptococcal associated disorders.

As used herein, “polypeptide” refers to full length proteins and fragments thereof.

As used herein, “peptide” refers to a fragment of the whole protein, whether chemically or biologically produced.

As used herein, “immunogenic” refers to an antigen or composition that elicits an immune response.

An “isolated nucleic acid molecule” refers to a polynucleotide molecule, in the form of a separate fragment or as a component of a larger nucleic acid construct, that has been separated from its source cell (including the chromosome it normally resides in) at least once in a substantially pure form. Nucleic acid molecules can be comprised of a wide variety of nucleotides and molecules well known in the art, including DNA, RNA, nucleic acid analogues, or any combination of these.

As used herein, “vector” refers to a polynucleotide assembly capable of directing expression and/or replication of the nucleic acid sequence of interest. Such assembly can, if desired, be included as a part of other components, such as a protein, lipid or lipoprotein coat, for delivery of the vector or for other purposes.

An “expression vector” refers to polynucleotide vector having at least a promoter sequence operably linked to the nucleic acid sequence of interest.

As used herein, a “promoter” refers to a nucleotide sequence that contains elements that direct the transcription of an operably linked nucleic acid sequence. At minimum, a promoter contains an RNA polymerase binding site. Promoter regions can also contain enhancer elements which by definition enhance transcription.

A. Hsp60 Genes and Polypeptides from Streptococcus pneumoniae and Streptococcus pyogenes

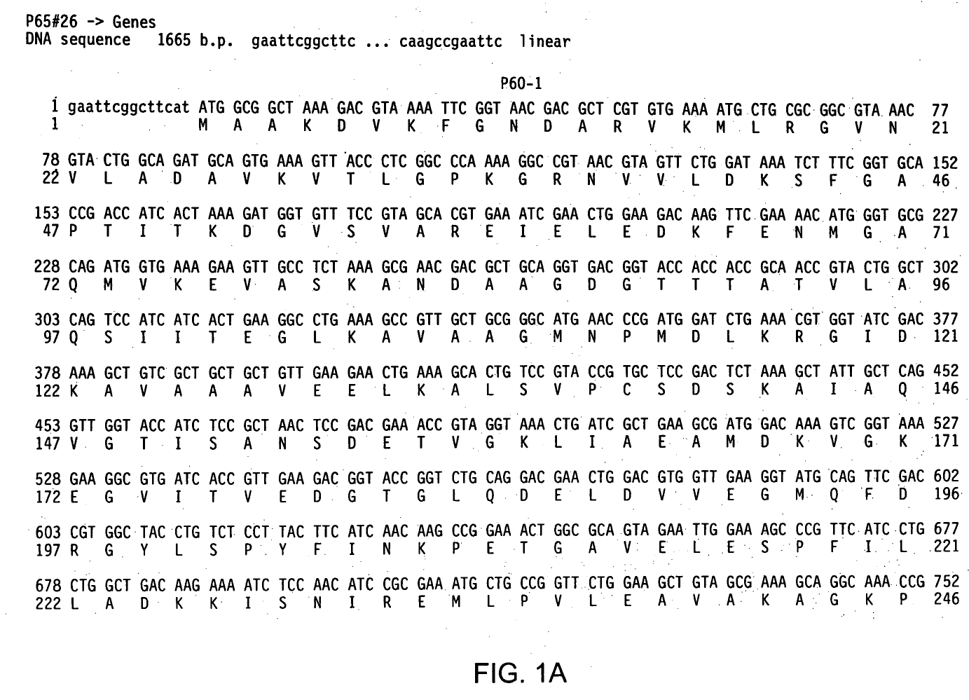

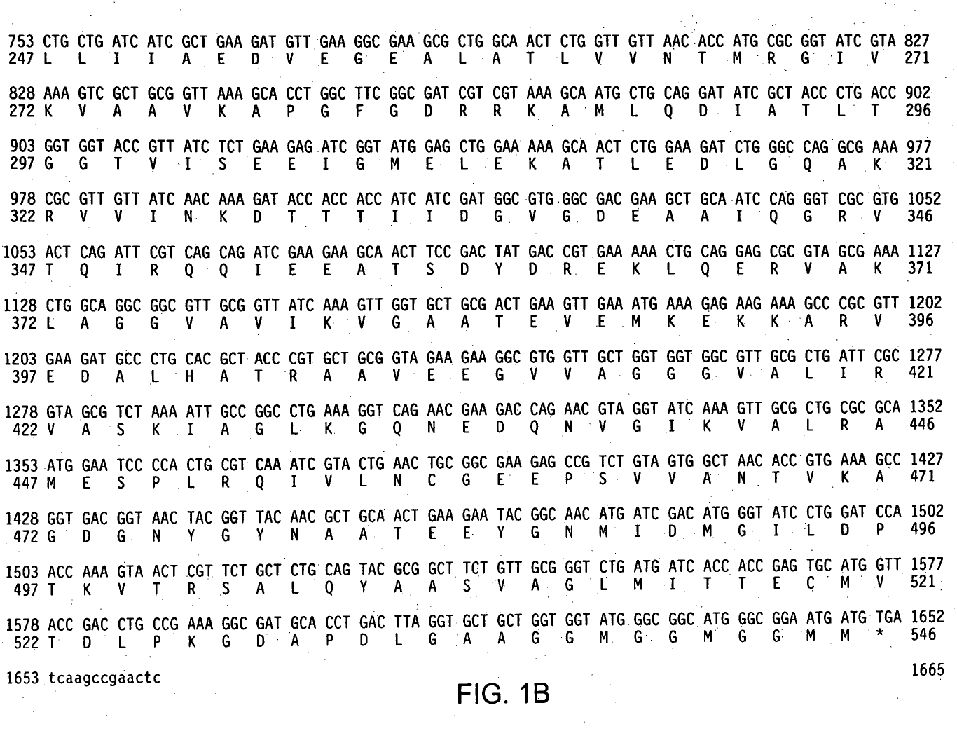

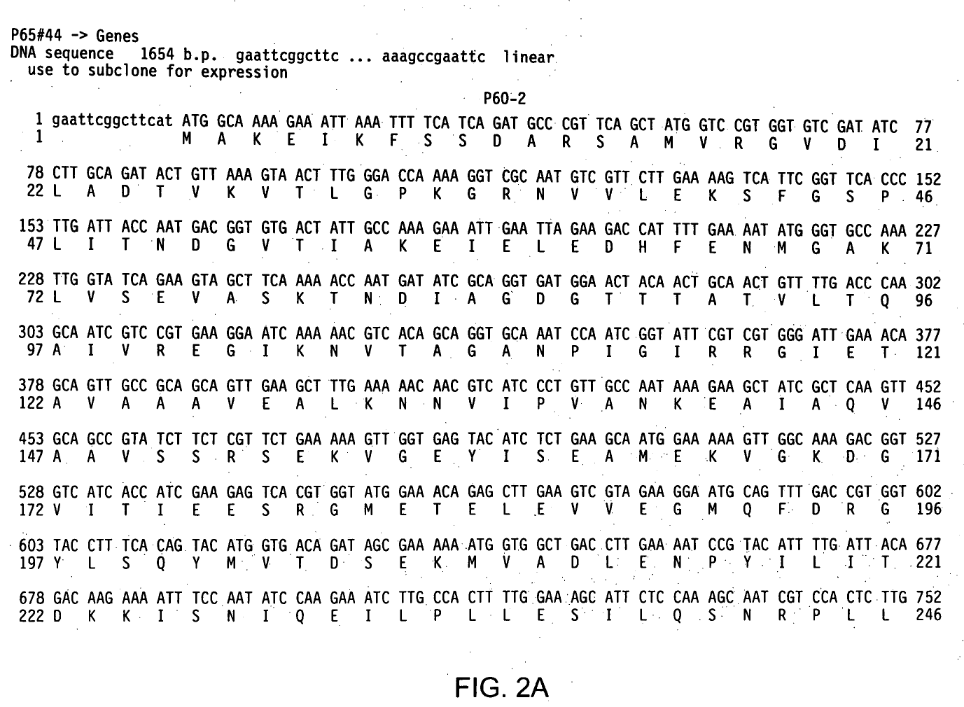

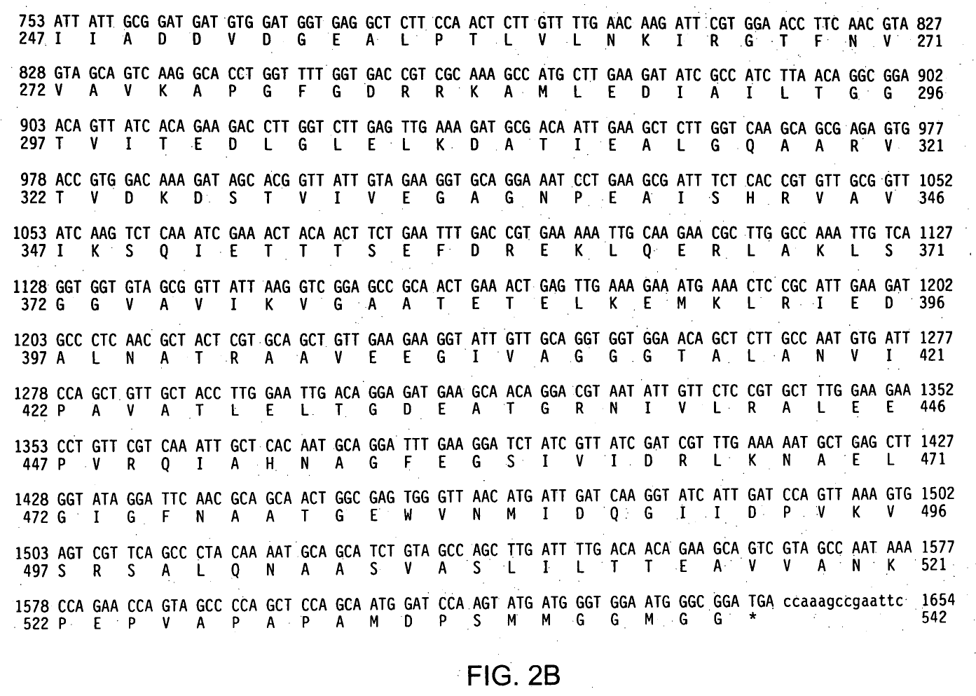

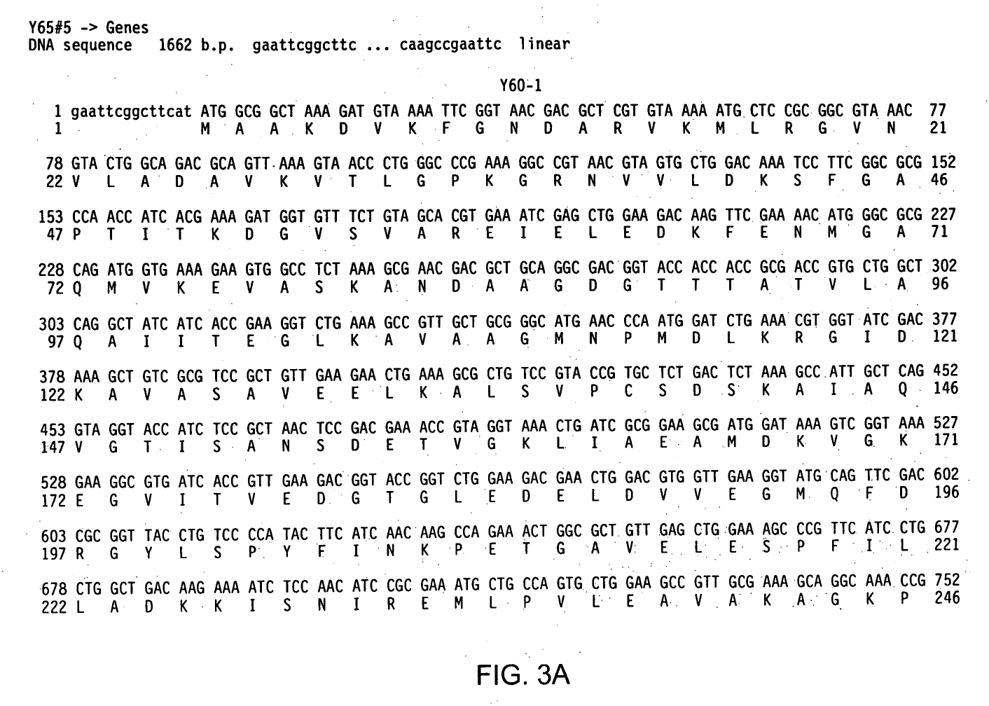

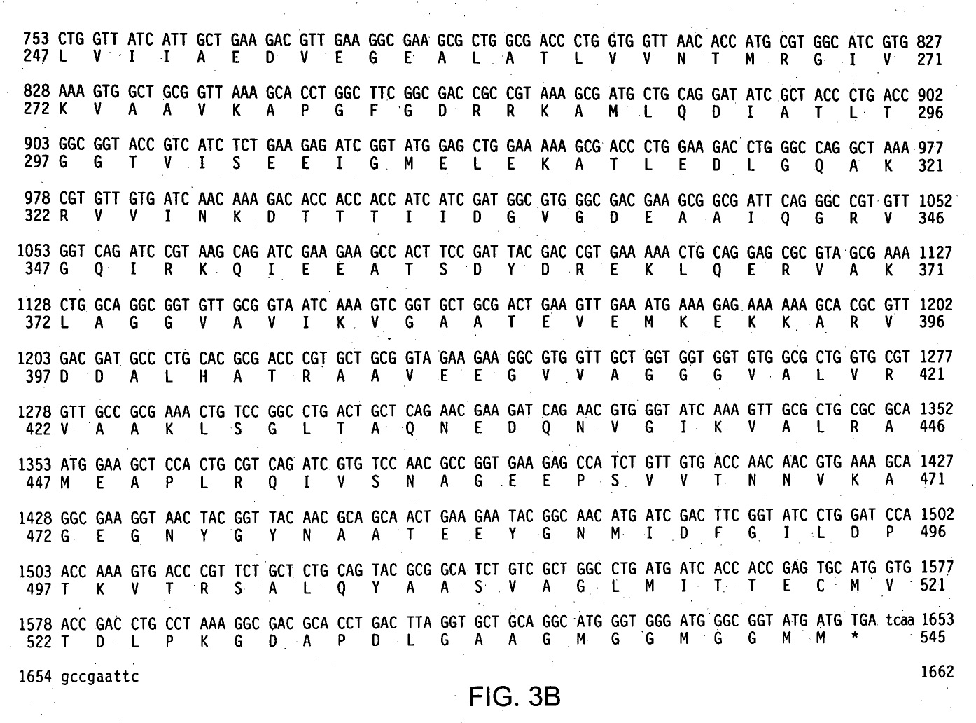

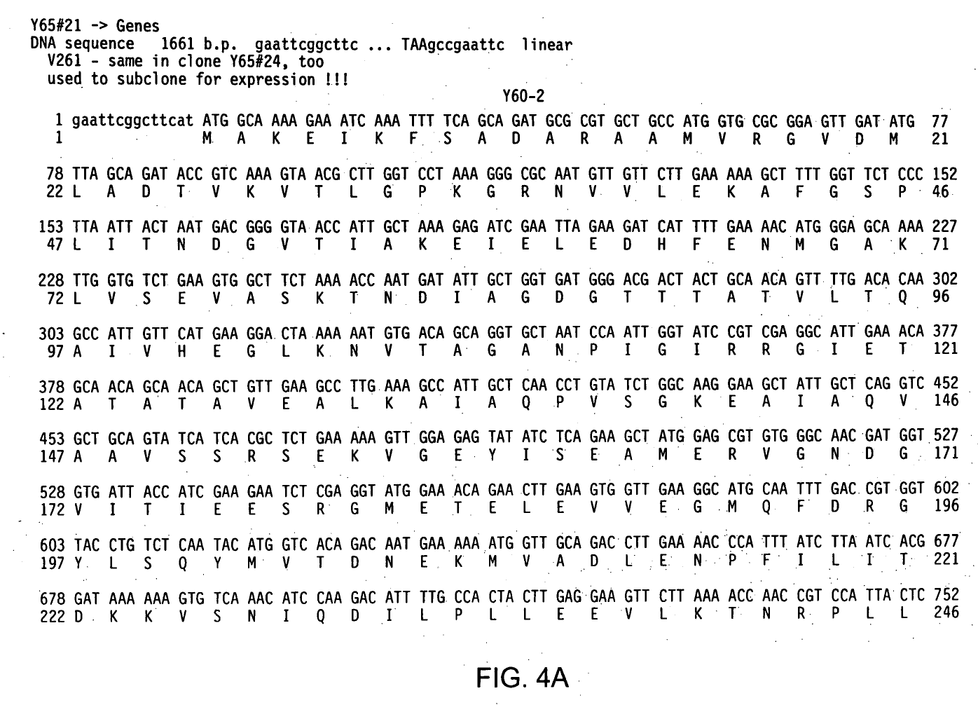

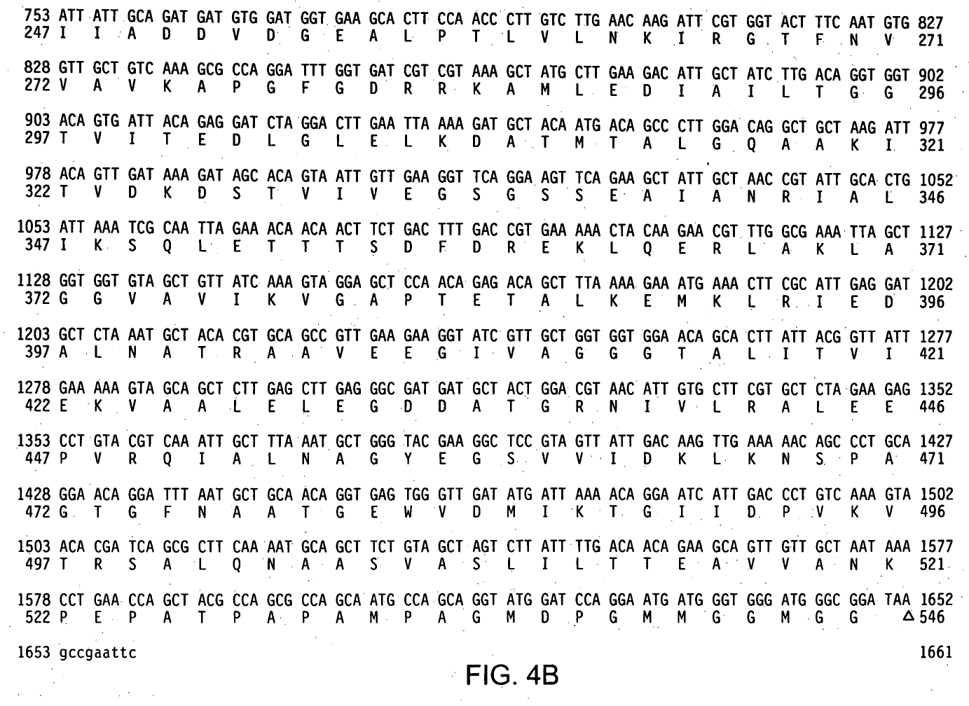

As used herein, Hsp60 refers to heat shock genes from the Hsp60 family of genes that produce heat shock proteins of approximately 60 kDa; the nucleotide and amino acid sequences of Hsp60 genes and gene products from Streptococcus pneumoniae and Streptococcus pyogenes are set forth in FIGS. 1-4 (SEQ ID NOS: 1-8; such sequences also include the PCR primers used to isolate the Hsp60 genes). Within the context of this invention it should be understood that Hsp60 includes wild-type/native protein sequences, as well as other variants (including alleles) and fragments of the native protein sequence. Briefly, such variants may result from natural polymorphisms or be synthesized by recombinant methodology or chemical synthesis, and differ from wild-type proteins by one or more amino acid substitutions, insertions, deletions, or the like. Further, in the region of homology to the native sequence, variants should preferably have at least 95% amino acid sequence homology, and within certain embodiments, greater than 97% or 98% homology. As will be appreciated by those of ordinary skill in the art, a nucleotide sequence encoding Hsp60 or variant may differ from the native sequences presented herein due to codon degeneracies, nucleotide polymorphisms, or nucleotide substitutions, deletions or insertions.

An “isolated nucleic acid molecule encoding Streptococcus Hsp60 ” refers to nucleic acid sequences that are capable of encoding Hsp60 polypeptides of Streptococcus, preferably Streptococcus pneumoniae or Streptococcus pyogenes. While several embodiments of such molecules are depicted in SEQ ID NOS: 1-4, it should be understood that within the context of the present invention, reference to one or more of these genes includes variants of the genes, that is, naturally occurring variants or sequences that are substantially similar to the genes (and, where appropriate, the protein (including peptides and polypeptides) that are encoded by the genes and their variants). As used herein, the nucleotide sequence is deemed to be “substantially similar” if: (a) the nucleotide sequence is derived from the coding region of a native gene of Streptococcus and maintains substantially the same biological activity (including, for example, portions of the sequence or allelic variations of the sequences discussed above); or (b) the nucleotide sequence is capable of hybridization to the nucleotide sequences of the present invention under high stringency (i.e., capable of selectively hybridizing to nucleotide sequences from Streptococcus); or (c) the nucleotide sequences are degenerate (i.e., sequences which code for the same amino acid using a different codon sequence) as a result of the genetic code to the nucleotide sequences defined in (a) or (b); or (d) is a complement of any of the sequences described in (a), (b) or (c)

One aspect of the present invention is the use of Streptococcus Hsp60 nucleotide sequences to produce recombinant proteins for immunizing an animal. Therefore, the use of any length of nucleic acid disclosed by the present invention (preferably 24 nucleotides or longer) which encodes a polypeptide or fragment thereof that is capable of binding to the major histocompatibility complex and eliciting or enhancing an immunogenic response is contemplated by this invention. Immunogenic response can be readily tested by known methods such as challenging a mouse or rabbit with the antigen of interest and thereafter collecting plasma and determining if the antibody of interest is present. Other assays particularly useful for the detection of T-cell responses include proliferation assays, T-cell cytotoxicity assays and assays for delayed hypersensitivity. In determining whether an antibody specific for the antigen of interest was produced by the animal, many diagnostic tools are available, for example, testing binding of labeled antigen to plasma derived antibodies, or using Enzyme-linked immunoassays with tag attached to the antigen of interest.

The Streptococcal Hsp60 genes of this invention can be obtained using a variety of methods. For example, a nucleic acid molecule can be obtained from a cDNA or genomic expression library by screening with an antibody or antibodies reactive to one or more of these Hsp60s (see, e.g., Sambrook et al., Molecular Cloning: A Laboratory Manual, Cold Spring Harbor, 1989; Ausubel et al., Current Protocols in Molecular Biology, Greene Publishing, 1987). Further, random-primed PCR can be employed (see, e.g., Methods in Enzymol. 254:275, 1995). In addition, variations of random-primed PCR can also be used, especially when a particular gene or gene family is desired. In one such method, one of the primers is a poly deoxy-thymine and the other is a degenerate primer based on the amino acid sequence or nucleotide sequence of related Hsps.

Other methods can also be used to obtain a nucleic acid molecule that encodes Streptococcal Hsp60. For example, a nucleic acid molecule can be obtained by using the sequence information provided herein to synthesize a probe which can be labeled, such as with a radioactive label, enzymatic label, protein label, fluorescent label, or the like, and hybridized to a genomic library or a cDNA library constructed in a phage, plasmid, phagemid, or other viral vector (see, e.g., Sambrook et al. (supra); Ausubel et al. (supra)). DNA representing RNA or genomic nucleic acid sequence can also be obtained by amplification using sets of primers complementary to 5′ and 3′ sequences of the cDNA sequence, such as presented in Example 1. For ease of cloning, restriction sites can also be incorporated into the primers.

Variants (including alleles) of the Hsp60 genes provided herein can be readily isolated from natural variants (e.g., polymorphisms, mutants), synthesized or constructed. Many methods have been developed for generating mutants (see generally Sambrook et al. (supra); Ausubel et al. (supra)). Briefly, preferred methods for generating nucleotide substitutions utilize an oligonucleotide that spans the base or bases to be mutated and contains the mutated base or bases. The oligonucleotide is hybridized to complementary single stranded nucleic acid and second strand synthesis is primed from the oligonucleotide. The double-stranded nucleic acid is prepared for transformation into host cells, such as E. coli, other prokaryotes, yeast or other eukaryotes. Standard screening and vector growth protocols are used to identify mutant sequences and obtain high yields.

Similarly, deletions and/or insertions of the Hsp60 gene can be constructed by any of a variety of known methods. For example, the gene can be digested with restriction enzymes and religated such that sequence is deleted or religated with additional sequence such that an insertion or large substitution is made. Other means of generating variant sequences, known in the art, can be employed, for examples see Sambrook et al. (supra) and Ausubel et al. (supra). Moreover, verification of variant sequences is typically accomplished by restriction enzyme mapping, sequence analysis or hybridization. Variants which encode a polypeptide that elicits an immunogenic response specific for Streptococcus are useful in the context of this invention.

As noted above, the present invention also provides isolated polypeptides. Within the context of the present invention, unless otherwise clear from the context, such polypeptides are understood to include the whole, or portions/fragments, of a gene product derived from one or more of the Streptococcal Hsp60 genes or derivatives thereof as discussed above. In one aspect of the present invention, the protein is encoded by a portion of a native gene or is encoded by a derivative of a native gene and the protein or fragment thereof elicits or enhances an immune response specific for Streptococcus.

A “purified” Hsp60 stress protein of the present invention is a heat shock protein of the Hsp60 family from Streptococcus pneumoniae or Streptococcus pyogenes that has been purified from its producing cell. For example, the Streptococcal Hsp60 polypeptides of the present invention can be purified by a variety of standard methods with or without a detergent purification step. For example, Streptococcal Hsp60 can be isolated by, among other methods, culturing suitable host and vector systems to produce recombinant Hsp60 (discussed further herein). Then, supernatants from such cell lines, or Hsp60 inclusions, or whole cells where the Hsp60 is not excreted into the supernatant, can be treated by a variety of purification procedures. For example, the Streptococcal Hsp60-containing composition can be applied to a suitable purification matrix such as an anti-Hsp60 antibody bound to a suitable support. Alternatively, anion or cation exchange resins, gel filtration or affinity, hydrophobic or reverse phase chromatography may be employed in order to purify the protein. The Hsp60 polypeptide can also be concentrated using commercially available protein concentration filters, such as an Amicon or Millipore Pellicon ultrafiltration unit, or by vacuum dialysis. In another alternative the supernatant can first be concentrated using one of the above mentioned protein concentration filters, followed by application of the concentrate to a suitable purification matrix such as those described above.

In one embodiment, the isolated Streptococcal Hsp60 s of the present invention are produced in a recombinant form, utilizing genetic manipulation techniques that are well known in the art. For example, Streptococcal Hsp60 can be expressed as a histidine-tagged molecule, permitting purification on a nickel-chelating matrix. Alternatively, other tags may be used, including FLAG and GST. The associated tag can then be removed in the last step of purification, for example, for certain vectors, His-tagged proteins may be incubated with thrombin, resulting in cleavage of a recognition sequence between the tag and the Hsp60 polypeptide (e.g., pET vectors from Invitrogen). Following purification of Streptococcal Hsp60 from a gram-negative bacterial host, whether tagged or not, it will be necessary to reduce the level of endotoxin in the Hsp60 preparation, as discussed above.

B. Vectors, Host Cells, and Expression of Streptococcal Hsp60

It is well known in the art that certain vectors (e.g., pUC) can be used for producing multiple copies of a nucleotide molecule of interest as well as being useful for genetic manipulation techniques (e.g., site-directed mutagenesis). See Sambrook (supra). Of particular interest to this disclosure are expression vectors. The expression vector includes transcriptional promoter/enhancer elements operably linked to the Streptococcal Hsp60 nucleic acid molecule. The expression vector may be composed of either deoxyribonucleic acids (“DNA”), ribonucleic acids (“RNA”), or a combination of the two (e.g., a DNA-RNA chimera). Optionally, the expression vector may include a polyadenylation sequence or one or more restriction sites. Additionally, depending on the host cell chosen and the expression vector employed, other genetic elements such as an origin of replication, additional nucleic acid restriction sites, enhancers, sequences conferring inducibility of transcription, and genes encoding proteins suitable for use as selectable or identifiable markers, may also be incorporated into the expression vectors described herein.

The manipulation and expression of Streptococcal Hsp60 genes can be accomplished by culturing host cells containing an expression vector capable of expressing the Hsp60 genes. Such vectors or vector constructs include either synthetic or cDNA-derived nucleic acid molecules or genomic DNA fragments encoding Streptococcal Hsp60 polypeptides, which are operably linked to suitable transcriptional or translational regulatory elements. Suitable regulatory elements within the expression vector can be derived from a variety of sources, including bacterial, fungal, viral, mammalian, insect, or plant genes. Selection of appropriate regulatory elements is dependent on the host cell chosen, and can be readily accomplished by one of ordinary skill in the art in light of the present specification. Examples of regulatory elements include a transcriptional promoter and enhancer or RNA polymerase binding sequence, a transcriptional terminator, and a ribosomal binding sequence, including a translation initiation signal.

Nucleic acid molecules that encode any of the Streptococcal Hsp60 polypeptides described above can be expressed by a wide variety of prokaryotic and eukaryotic host cells, including bacterial, mammalian, yeast or other fungi, viral, insect, and plant cells. The selection of a host cell may also assist the production of glycosolated or non-glycosolated Hsp60s, depending upon the desires of the user. Methods for transforming or transfecting such cells to express nucleic acids are well known in the art (see, e.g., Itakura et al., U.S. Pat. No. 4,704,362; Hinnen et al., PNAS USA 75:1929-1933, 1978; Murray et al., U.S. Pat. No. 4,801,542; Upshall et al., U.S. Pat. No. 4,935,349; Hagen et al., U.S. Pat. No. 4,784,950; Axel et al., U.S. Pat. No. 4,399,216; Goeddel et al., U.S. Pat. No. 4,766,075; and Sambrook et al., Molecular Cloning: A Laboratory Manual, 2nd edition, Cold Spring Harbor Laboratory Press, 1989; for plant cells see Czako and Marton, Plant Physiol. 104:1067-1071, 1994; Paszkowski et al., Biotech. 24:387-392, 1992).

Bacterial host cells suitable for carrying out the present invention include E. coli, such as E. coli DH5α (Stratagene, La Jolla, Calif.), M. leprae, M. tuberculosis, M. bovis, B. subtilis, Salmonella typhimurium, and various species within the genera Pseudomonas, Streptomyces, Streptococcus, and Staphylococcus, as well as many other bacterial species well known to one of ordinary skill in the art.

Bacterial expression vectors preferably comprise a promoter, which functions in the host cell, one or more selectable phenotypic markers, and a bacterial origin of replication. Representative promoters include the β-lactamase (penicillinase) and lactose promoter system (see Chang et al., Nature 275:615, 1978), the T7 RNA polymerase promoter (Studier et al., Meth. Enzymol. 185:60-89, 1990), the lambda promoter (Elvin et al., Gene 87:123-126, 1990), the trp promoter (Nichols and Yanofsky, Meth. in Enzymology 101:155, 1983) and the tac promoter (Russell et al., Gene 20: 231, 1982). Representative selectable markers include various antibiotic resistance markers such as the kanamycin or ampicillin resistance genes. Many plasmids suitable for transforming host cells are well known in the art, including among others, pBR322 (see Bolivar et al., Gene 2:95, 1977), the pUC plasmids pUC18, pUC19, pUC118, pUC119 (see Messing, Meth. in Enzymology 101:20-77, 1983; Vieira and Messing, Gene 19:259-268, 1982), and pNH8A, pNH16a, pNH18a, and BLUESCRIPT M13 (Stratagene, La Jolla, Calif.).

Fungal host cells suitable for carrying out the present invention include, among others, Saccharomyces pombe, Saccharomyces cerevisiae, the genera Pichia or Kluyveromyces and various species of the genus Aspergillus (McKnight et al., U.S. Pat. No. 4,935,349). Suitable expression vectors for yeast and fungi include, among others, YCp50 (ATCC No. 37419) for yeast, and the amdS cloning vector pV3 (Tumbull, Bio/Technology 7:169, 1989), YRp7 (Struhl et al., Proc. Natl. Acad. Sci. USA 76:1035-1039, 1978), YEp13 (Broach et al., Gene 8:121-133, 1979), pJDB249 and pJDB219 (Beggs, Nature 275:104-108, 1978) and derivatives thereof.

Preferred promoters for use in yeast include promoters from yeast glycolytic genes (Hitzeman et al., J. Biol. Chem. 255:12073-12080, 1980; Alber and Kawasaki, J. Mol. Appl. Genet. 1:419-434, 1982) or alcohol dehydrogenase genes (Young et al., in Genetic Engineering of Microorganisms for Chemicals, Hollaender et al. (eds.), p. 355, Plenum, New York, 1982; Ammerer, Meth. Enzymol. 101:192-201, 1983). Examples of useful promoters for fungi vectors include those derived from Aspergillus nidulans glycolytic genes, such as the adh3 promoter (McKnight et al., EMBO J. 4:2093-2099, 1985). The expression units may also include a transcriptional terminator. An example of a suitable terminator is the adh3 terminator (McKnight et al., ibid., 1985).

As with bacterial vectors, the yeast vectors will generally include a selectable marker, which may be one of any number of genes that exhibit a dominant phenotype for which a phenotypic assay exists to enable transformants to be selected. Preferred selectable markers include those that complement host cell auxotrophy, provide antibiotic resistance or enable a cell to utilize specific carbon sources, and include leu2 (Broach et al., ibid.), ura3 (Botstein et al., Gene 8:17, 1979), or his3 (Struhl et al., ibid.). Another suitable selectable marker is the cat gene, which confers chloramphenicol resistance on yeast cells.

Techniques for transforming fungi are well known in the literature, and have been described, for instance, by Beggs (ibid.), Hinnen et al. (Proc. Natl. Acad. Sci. USA 75:1929-1933, 1978), Yelton et al. (Proc. Natl. Acad. Sci. USA 81:1740-1747, 1984), and Russell (Nature 301:167-169, 1983). The genotype of the host cell may contain a genetic defect that is complemented by the selectable marker present on the expression vector. Choice of a particular host and selectable marker is well within the level of ordinary skill in the art in light of the present specification.

Protocols for the transformation of yeast are also well known to those of ordinary skill in the art. For example, transformation may be readily accomplished either by preparation of spheroplasts of yeast with DNA (see Hinnen et al., PNAS USA 75:1929, 1978) or by treatment with alkaline salts such as LiCl (see Itoh et al., J. Bacteriology 153:163, 1983). Transformation of fungi may also be carried out using polyethylene glycol as described by Cullen et al. (Bio/Technology 5:369, 1987).

Viral vectors include those that comprise a promoter that directs the expression of an isolated nucleic acid molecule that encodes a Streptococcal Hsp60 as described above. A wide variety of promoters may be utilized within the context of the present invention, including for example, promoters such as MoMLV LTR, RSV LTR, Friend MuLV LTR, adenoviral promoter (Ohno et al., Science 265: 781-784, 1994), neomycin phosphotransferase promoter/enhancer, late parvovirus promoter (Koering et al., Hum. Gene Therap. 5:457-463, 1994), Herpes TK promoter, SV40 promoter, metallothionein IIa gene enhancer/promoter, cytomegalovirus immediate early promoter, and the cytomegalovirus immediate late promoter. The promoter may also be a tissue-specific promoter (see e.g., WO 91/02805; EP 0,415,731; and WO 90/07936). In addition to the above-noted promoters, other viral-specific promoters (e.g., retroviral promoters (including those noted above, as well as others such as HIV promoters), hepatitis, herpes (e.g., EBV), and bacterial, fungal or parasitic-specific (e.g., malarial-specific) promoters may be utilized in order to target a specific cell or tissue which is infected with a virus, bacteria, fungus or parasite.

Thus, Streptococcal Hsp60 polypeptides of the present invention may be expressed from a variety of viral vectors, including for example, herpes viral vectors (e.g., U.S. Pat. No. 5,288,641), adenoviral vectors (e.g., WO 94/26914, WO 93/9191; Kolls et al., PNAS 91(1):215-219, 1994; Kass-Eisler et al., PNAS 90(24):11498-502, 1993; Guzman et al., Circulation 88(6):2838-48, 1993; Guzman et al., Cir. Res. 73(6):1202-1207, 1993; Zabner et al., Cell 75(2):207-216, 1993; Li et al., Hum Gene Ther. 4(4):403-409, 1993; Caillaud et al., Eur. J. Neurosci. 5(10):1287-1291, 1993; Vincent et al., Nat. Genet. 5(2):130-134, 1993; Jaffe et al., Nat. Genet. 1(5):372-378, 1992; and Levrero et al., Gene 101(2):195-202, 1991), adenovirus-associated viral vectors (Flotte et al., PNAS 90(22):10613-10617, 1993), baculovirus vectors, parvovirus vectors (Koering et al., Hum. Gene Therap. 5:457-463, 1994), pox virus vectors (Panicali and Paoletti, PNAS 79:4927-4931, 1982; and Ozaki et al., Biochem. Biophys. Res. Comm. 193(2):653-660, 1993), and retroviruses (e.g., EP 0,415,731; WO 90/07936; WO 91/0285, WO 94/03622; WO 93/25698; WO 93/25234; U.S. Pat. No. 5,219,740; WO 93/11230; WO 93/10218. Within various embodiments, either the viral vector itself or a viral particle which contains the viral vector may be utilized in the methods and compositions described below.

Mammalian cells suitable for carrying out the present invention include, among others: PC12 (ATCC No. CRL1721), N1E-115 neuroblastoma, SK-N-BE(2)C neuroblastoma, SHSY5 adrenergic neuroblastoma, NS20Y and NG108-15 murine cholinergic cell lines, or rat F2 dorsal root ganglion line, COS (e.g., ATCC No. CRL 1650 or 1651), BHK (e.g., ATCC No. CRL 6281; BHK 570 cell line (deposited with the American Type Culture Collection under accession number CRL 10314), CHO (ATCC No. CCL 61), HeLa (e.g., ATCC No. CCL 2), 293 (ATCC No. 1573; Graham et al., J. Gen. Virol. 36:59-72, 1977) and NS-1 cells. Other mammalian cell lines may be used within the present invention, including Rat Hep I (ATCC No. CRL 1600), Rat Hep II (ATCC No. CRL 1548), TCMK (ATCC No. CCL 139), Human lung (ATCC No. CCL 75.1), Human hepatoma (ATCC No. HTB-52), Hep G2 (ATCC No. HB 8065), Mouse liver (ATCC No. CCL 29.1), NCTC 1469 (ATCC No. CCL 9.1), SP2/0-Ag14 (ATCC No. 1581), HIT-T15 (ATCC No. CRL 1777), and RINm 5AHT2B (Orskov and Nielson, FEBS 229(1):175-178, 1988).

Mammalian expression vectors for use in carrying out the present invention include a promoter capable of directing the transcription of a cloned gene or cDNA. Preferred promoters include viral promoters and cellular promoters. Viral promoters include the cytomegalovirus immediate early promoter (Boshart et al., Cell 41:521-530, 1985), cytomegalovirus immediate late promoter, SV40 promoter (Subramani et al., Mol. Cell. Biol. 1:854-864, 1981), MMTV LTR, RSV LTR, metallothionein-1, adenovirus E1a. Cellular promoters include the mouse metallothionein-1 promoter (Palmiter et al., U.S. Pat. No. 4,579,821), action promoters, a mouse V_{H} promoter (Bergman et al., Proc. Natl. Acad. Sci. USA 81:7041-7045, 1983; Grant et al., Nucl. Acids Res. 15:5496, 1987) and a mouse V_{H} promoter (Loh et al., Cell 33:85-93, 1983). The choice of promoter will depend, at least in part, upon the level of expression desired or the recipient cell line to be transfected.

Such expression vectors can also contain a set of RNA splice sites located downstream from the promoter and upstream from the DNA sequence encoding the peptide or protein of interest. Preferred RNA splice sites may be obtained from adenovirus and/or immunoglobulin genes. Also contained in the expression vectors is a polyadenylation signal located downstream of the coding sequence of interest. Suitable polyadenylation signals include the early or late polyadenylation signals from SV40 (Kaufman and Sharp, ibid.), the polyadenylation signal from the Adenovirus 5 EIB region and the human growth hormone gene terminator (DeNoto et al., Nuc. Acids Res. 9:3719-3730, 1981). The expression vectors may include a noncoding viral leader sequence, such as the Adenovirus 2 tripartite leader, located between the promoter and the RNA splice sites. Preferred vectors may also include enhancer sequences, such as the SV40 enhancer. Expression vectors may also include sequences encoding the adenovirus VA RNAs. Suitable expression vectors can be obtained from commercial sources (e.g., Stratagene, La Jolla, Calif.).

Vector constructs comprising cloned DNA sequences can be introduced into cultured mammalian cells by, for example, calcium phosphate-mediated transfection (Wigler et al., Cell 14:725, 1978; Corsaro and Pearson, Somatic Cell Genetics 7:603, 1981; Graham and Van der Eb, Virology 52:456, 1973), electroporation (Neumann et al., EMBO J. 1:841-845, 1982), or DEAE-dextran mediated transfection (Ausubel et al. (eds.), Current Protocols in Molecular Biology, John Wiley and Sons, Inc., NY, 1987). See generally Sambrook et al. (supra). To identify cells that have stably integrated the cloned DNA, a selectable marker is generally introduced into the cells along with the gene or cDNA of interest. Preferred selectable markers for use in cultured mammalian cells include genes that confer resistance to drugs, such as neomycin, hygromycin, and methotrexate. The selectable marker may be an amplifiable selectable marker. Preferred amplifiable selectable markers are the DHFR gene and the neomycin resistance gene. Selectable markers are reviewed by Thilly (Mammalian Cell Technology, Butterworth Publishers, Stoneham, Mass.).

Mammalian cells containing a suitable vector are allowed to grow for a period of time, typically 1-2 days, to begin expressing the DNA sequence(s) of interest. Drug selection is then applied to select for growth of cells that are expressing the selectable marker in a stable fashion. For cells that have been transfected with an amplifiable, selectable marker the drug concentration may be increased in a stepwise manner to select for increased copy number of the cloned sequences, thereby increasing expression levels. Cells expressing the introduced sequences are selected and screened for production of the protein of interest in the desired form or at the desired level. Cells that satisfy these criteria can then be cloned and scaled up for production.

Numerous insect host cells known in the art can also be useful within the present invention, in light of the subject specification. For example, the use of baculoviruses as vectors for expressing heterologous DNA sequences in insect cells has been reviewed by Atkinson et al. (Pestic. Sci. 28:215-224, 1990).

Numerous plant host cells known in the art can also be useful within the present invention, in light of the subject specification. For example, the use of Agrobacterium rhizogenes as vectors for expressing genes in plant cells has been reviewed by Sinkar et al., J. Biosci. (Bangalore) 11:47-58, 1987.

Upon expression of the Streptococcal Hsp60 polypeptides or fragments thereof in the host cells, the polypeptide or peptide may be preliminarily released and/or isolated from the host cell utilizing methods such as those discussed previously herein.

As noted above, depending on the host cell in which one desires to express Hsp60, the gene encoding the protein is introduced into an expression vector comprising a promoter that is active in the host cell. Other components of the expression unit such as transcribed but not translated sequences at the ends of the coding region may also be selected according to the particular host utilized. In some cases, it may be necessary to introduce artificially an intervening sequence to ensure high level expression. Expression can be monitored by SDS-PAGE and staining, if expression levels are sufficiently high. Additionally, if the protein is produced with a tag, detection by anti-tag antibody can be carried out and if produced with no tag, detection by anti-Hsp60 antibody that does not recognize homologous proteins of the host may be employed. Further, any method known in the art for protein identification may be utilized to this end (e.g., a high resolution electrophoretic method or 2D electrophoresis).

C. Preparation of Antibodies Against the Hsp60 Polypeptides of the Present Invention

In another aspect, the proteins of the present invention are utilized to prepare specifically binding antibodies (i.e., binding partners). Accordingly, the present invention also provides such antibodies. Within the context of the present invention, the term “antibodies” includes polyclonal antibodies, monoclonal antibodies, anti-idiotypic antibodies, fragments thereof such as F(ab′)₂ and Fab fragments, and recombinantly or synthetically produced binding partners. Such binding partners incorporate the variable regions that permit a monoclonal antibody to specifically bind, which means an antibody able to selectively bind to a peptide produced from one of the Streptococcal Hsp60 genes of the invention. The affinity of a monoclonal antibody or binding partner can be readily determined by one of ordinary skill in the art (see Scatchard, Ann. N. Y. Acad. Sci. 51:660-672, 1949).

Polyclonal antibodies can be readily generated by one of ordinary skill in the art from a variety of warm-blooded animals such as horses, cows, goats, sheep, dogs, chickens, turkeys, rabbits, mice, or rats. Briefly, the desired protein or peptide is utilized to immunize the animal through intraperitoneal, intramuscular, intraocular, or subcutaneous injections. The immunogenicity of the protein or peptide of interest may be increased through the use of an adjuvant such as Freund's complete or incomplete adjuvant. Following several booster immunizations, small samples of serum are collected and tested for reactivity to the desired protein or peptide.

Particularly preferred polyclonal antisera give a signal that is at least three times greater than background. Once the titer of the animal has reached a plateau in terms of its reactivity to the protein, larger quantities of polyclonal antisera may be readily obtained either by weekly bleedings, or by exsanguinating the animal.

Monoclonal antibodies can also be readily generated using well-known techniques (see U.S. Pat. Nos. RE 32,011, 4,902,614, 4,543,439, and 4,411,993; see also Monoclonal Antibodies, Hybridomas: A New Dimension in Biological Analyses, Plenum Press, Kennett, McKeam, and Bechtol (eds.), 1980, and Antibodies: A Laboratory Manual, Harlow and Lane (eds.), Cold Spring Harbor Laboratory Press, 1988). Briefly, in one embodiment, a subject animal such as a rat or mouse is injected with a desired protein or peptide. If desired, various techniques may be utilized in order to increase the resultant immune response generated by the protein, in order to develop greater antibody reactivity. For example, the desired protein or peptide may be coupled to another protein such as ovalbumin or keyhole limpet hemocyanin (KLH), or through the use of adjuvants such as Freund's complete or incomplete adjuvant. The initial elicitation of an immune response, may preferably be through intraperitoneal, intramuscular, intraocular, or subcutaneous routes.

Between one and three weeks after the initial immunization, the animal may be reimmunized. The animal may then be test bled and the serum tested for binding to the desired antigen using assays as described above. Additional immunizations may also be accomplished until the animal has reached a plateau in its reactivity to the desired protein or peptide. The animal may then be given a final boost of the desired protein or peptide, and three to four days later sacrificed. At this time, the spleen and lymph nodes may be harvested and disrupted into a single cell suspension by passing the organs through a mesh screen or by rupturing the spleen or lymph node membranes which encapsulate the cells. Within one embodiment the red cells are subsequently lysed by the addition of a hypotonic solution, followed by immediate return to isotonicity.

Within another embodiment, suitable cells for preparing monoclonal antibodies are obtained through the use of in vitro immunization techniques. Briefly, an animal is sacrificed, and the spleen and lymph node cells are removed as described above. A single cell suspension is prepared, and the cells are placed into a culture containing a form of the protein or peptide of interest that is suitable for generating an immune response as described above. Subsequently, the lymphocytes are harvested and fused as described below.

Cells that are obtained through the use of in vitro immunization or from an immunized animal as described above may be immortalized by transfection with a virus such as the Epstein-Barr Virus (EBV). (See Glasky and Reading, Hybridoma 8(4):377-389, 1989.) Alternatively, within a preferred embodiment, the harvested spleen and/or lymph node cell suspensions are fused with a suitable myeloma cell in order to create a “hybridoma” which secretes monoclonal antibodies. Suitable myeloma lines are preferably defective in the construction or expression of antibodies, and are additionally syngeneic with the cells from the immunized animal. Many such myeloma cell lines are well known in the art and may be obtained from sources such as the American Type Culture Collection (ATCC), 10801 University Boulevard, Manassas, Va. 20110-2209 (see Catalogue of Cell Lines & Hybridomas, 6th ed., ATCC, 1988). Representative myeloma lines include: for humans, UC 729-6 (ATCC No. CRL 8061), MC/CAR-Z2 (ATCC No. CRL 8147), and SKO-007 (ATCC No. CRL 8033); for mice, SP2/0-Ag14 (ATCC No. CRL 1581), and P3X63Ag8 (ATCC No. TIB 9); and for rats, Y3-Ag1.2.3 (ATCC No. CRL 1631), and YB2/0 (ATCC No. CRL 1662). Particularly preferred fusion lines include NS-1 (ATCC No. TIB 18) and P3X63—Ag 8.653 (ATCC No. CRL 1580), which may be utilized for fusions with either mouse, rat, or human cell lines. Fusion between the myeloma cell line and the cells from the immunized animal can be accomplished by a variety of methods, including the use of polyethylene glycol (PEG) (see Antibodies: A Laboratory Manual, Harlow and Lane, supra) or electrofusion. (See Zimmerman and Vienken, J. Membrane Biol. 67:165-182, 1982.)

Following the fusion, the cells are placed into culture plates containing a suitable medium, such as RPMI 1640 or DMEM (Dulbecco's Modified Eagles Medium, JRH Biosciences, Lenexa, Kans.). The medium may also contain additional ingredients, such as Fetal Bovine Serum (FBS, e.g., from Hyclone, Logan, Utah, or JRH Biosciences), thymocytes that were harvested from a baby animal of the same species as was used for immunization, or agar to solidify the medium. Additionally, the medium should contain a reagent which selectively allows for the growth of fused spleen and myeloma cells. Particularly preferred is the use of HAT medium (hypoxanthine, aminopterin, and thymidine) (Sigma Chemical Co., St. Louis, Mo.). After about seven days, the resulting fused cells or hybridomas may be screened in order to determine the presence of antibodies which recognize the desired antigen. Following several clonal dilutions and reassays, hybridoma producing antibodies that bind to the protein of interest can be isolated.

Other techniques may also be utilized to construct monoclonal antibodies. (See Huse et al., “Generation of a Large Combinational Library of the Immunoglobulin Repertoire in Phage Lambda,” Science 246:1275-1281, 1989; see also Sastry et al., “Cloning of the Immunological Repertoire in Escherichia coli for Generation of Monoclonal Catalytic Antibodies: Construction of a Heavy Chain Variable Region-Specific cDNA Library,” Proc. Natl. Acad. Sci. USA 86:5728-5732, 1989; see also Alting-Mees et al., “Monoclonal Antibody Expression Libraries: A Rapid Alternative to Hybridomas,” Strategies in Molecular Biology 3:1-9, 1990; these references describe a commercial system available from Stratagene, La Jolla, Calif., which enables the production of antibodies through recombinant techniques.) Briefly, mRNA is isolated from a B cell population and utilized to create heavy and light chain immunoglobulin cDNA expression libraries in the λIMMUNOZAP(H) and λIMMUNOZAP(L) vectors. These vectors may be screened individually or co-expressed to form Fab fragments or antibodies (see Huse et al. (supra); see also Sastry et al. (supra)). Positive plaques can subsequently be converted to a non-lytic plasmid which allows high level expression of monoclonal antibody fragments from E. coli.

Similarly, binding partners can also be constructed utilizing recombinant DNA techniques to incorporate the variable regions of a gene that encodes a specifically binding antibody. The construction of these binding partners can be readily accomplished by one of ordinary skill in the art given the disclosure provided herein. (See Larrick et al., “Polymerase Chain Reaction Using Mixed Primers: Cloning of Human Monoclonal Antibody Variable Region Genes From Single Hybridoma Cells,” Biotechnology 7:934-938, 1989; Riechmann et al., “Reshaping Human Antibodies for Therapy,” Nature 332:323-327, 1988; Roberts et al., “Generation of an Antibody with Enhanced Affinity and Specificity for its Antigen by Protein Engineering,” Nature 328:731-734, 1987; Verhoeyen et al., “Reshaping Human Antibodies: Grafting an Antilysozyme Activity,” Science 239:1534-1536, 1988; Chaudhary et al., “A Recombinant Immunotoxin Consisting of Two Antibody Variable Domains Fused to Pseudomonas Exotoxin,” Nature 339:394-397, 1989; see also U.S. Pat. No. 5,132,405 entitled “Biosynthetic Antibody Binding Sites.”) Briefly, in one embodiment, DNA segments encoding the desired protein or peptide of interest-specific antigen binding domains are amplified from hybridomas that produce a specifically binding monoclonal antibody, and are inserted directly into the genome of a cell that produces human antibodies. (See Verhoeyen et al. (supra); see also Reichmann et al. (supra)). This technique allows the antigen-binding site of a specifically binding mouse or rat monoclonal antibody to be transferred into a human antibody. Such antibodies are preferable for therapeutic use in humans because they are not as antigenic as rat or mouse antibodies.

In an alternative embodiment, genes that encode the variable region from a hybridoma producing a monoclonal antibody of interest are amplified using oligonucleotide primers for the variable region. These primers may be synthesized by one of ordinary skill in the art, or may be purchased from commercially available sources. For instance, primers for mouse and human variable regions including, among others, primers for V_{Ha}, V_{Hb}, V_{Hc}, V_{Hd}, C_{H1}, V_{L} and C_{L} regions, are available from Stratagene (La Jolla, Calif.). These primers may be utilized to amplify heavy or light chain variable regions, which may then be inserted into vectors such as IMMUNOZAP™(H) or IMMUNOZAP™(L) (Stratagene), respectively. These vectors may then be introduced into E. coli for expression. Utilizing these techniques, large amounts of a single-chain polypeptide containing a fusion of the V_{H} and V_{L} domains may be produced (see Bird et al., Science 242:423-426, 1988).

Monoclonal antibodies and other binding partners can be produced in a number of host systems, including tissue cultures, bacteria, eukaryotic cells, plants and other host systems known in the art.

Once suitable antibodies or binding partners have been obtained, they may be isolated or purified by many techniques well known to those of ordinary skill in the art (see Antibodies: A Laboratory Manual, Harlow and Lane (supra)). Suitable techniques include peptide or protein affinity columns, HPLC or RP-HPLC, purification on protein A or protein G columns, or any combination of these techniques. Within the context of the present invention, the term “isolated” as used to define antibodies or binding partners means “substantially free of other blood components.”

The binding partners of the present invention have many uses. For example, antibodies can be utilized in flow cytometry to identify cells bearing such a protein. Briefly, in order to detect the protein or peptide of interest on cells, the cells are incubated with a labeled monoclonal antibody which specifically binds to the protein of interest, followed by detection of the presence of bound antibody. Labels suitable for use within the present invention are well known in the art including, among others, flourescein isothiocyanate (FITC), phycoerythrin (PE), horse radish peroxidase (HRP), and colloidal gold. Particularly preferred for use in flow cytometry is FITC, which may be conjugated to purified antibody according to the method of Keltkamp in “Conjugation of Fluorescein Isothiocyanate to Antibodies. I. Experiments on the Conditions of Conjugation,” Immunology 18:865-873, 1970. (See also Keltkamp, “Conjugation of Fluorescein Isothiocyanate to Antibodies. II. A Reproducible Method,” Immunology 18:875-881, 1970; Goding, “Conjugation of Antibodies with Fluorochromes: Modification to the Standard Methods,” J. Immunol. Methods 13:215-226, 1970.) The antibodies can also be used to target drugs to Streptococcus as well as a diagnostic for determining Streptococcal infection.

D. Assays that Utilize the Hsp60 Polypeptides, or Antibodies Thereto, of the Present Invention

A variety of assays can be utilized in order to detect the Hsp60 polypeptides from Streptococcus pneumoniae and Streptococcus pyogenes of the present invention, or antibodies that specifically bind to such Hsp60 polypeptides. Exemplary assays are described in detail in Antibodies: A Laboratory Manual, Harlow and Lane (eds.), Cold Spring Harbor Laboratory Press, 1988. Representative examples of such assays include: countercurrent immuno-electrophoresis (CIEP), radioimmunoassays, radioimmunoprecipitations, enzyme-linked immuno-sorbent assays (ELISA), dot blot assays, inhibition or competition assays, and sandwich assays, immunostick (dipstick) assays, simultaneous immunoassays, immunochromatographic assays, immunofiltration assays, latex bead agglutination assays, immunofluorescent assays, biosensor assays, and low-light detection assays (see U.S. Pat. Nos. 4,376,110 and 4,486,530; see also Antibodies: A Laboratory Manual (supra)).

A fluorescent antibody test (FA-test) uses a fluorescently labeled antibody able to bind to one of the proteins of the invention. For detection, visual determinations are made by a technician using fluorescence microscopy, yielding a qualitative result. In one embodiment, this assay is used for the examination of tissue samples or histological sections.

In latex bead agglutination assays, antibodies to one or more of the proteins of the present invention are conjugated to latex beads. The antibodies conjugated to the latex beads are then contacted with a sample under conditions permitting the antibodies to bind to desired proteins in the sample, if any. The results are then read visually, yielding a qualitative result. In one embodiment, this format can be used in the field for on-site testing.

Enzyme immunoassays (EIA) include a number of different assays able to utilize the antibodies provided by the present invention. For example, a heterogeneous indirect EIA uses a solid phase coupled with an antibody of the invention and an affinity purified, anti-IgG immunoglobulin preparation. Preferably, the solid phase is a polystyrene microtiter plate. The antibodies and immunoglobulin preparation are then contacted with the sample under conditions permitting antibody binding, which conditions are well known in the art. The results of such an assay can be read visually, but are preferably read using a spectrophotometer, such as an ELISA plate reader, to yield a quantitative result. An alternative solid phase EIA format includes plastic-coated ferrous metal beads able to be moved during the procedures of the assay by means of a magnet. Yet another alternative is a low-light detection immunoassay format. In this highly sensitive format, the light emission produced by appropriately labeled bound antibodies are quantitated automatically. Preferably, the reaction is performed using microtiter plates.

In an alternative embodiment, a radioactive tracer is substituted for the enzyme mediated detection in an EIA to produce a radioimmunoassay (RIA).

In a capture-antibody sandwich enzyme assay, the desired protein is bound between an antibody attached to a solid phase, preferably a polystyrene microtiter plate, and a labeled antibody. Preferably, the results are measured using a spectrophotometer, such as an ELISA plate reader.

In a sequential assay format, reagents are allowed to incubate with the capture antibody in a step wise fashion. The test sample is first incubated with the capture antibody. Following a wash step, an incubation with the labeled antibody occurs. In a simultaneous assay, the two incubation periods described in the sequential assay are combined. This eliminates one incubation period plus a wash step.

A dipstick/immunostick format is essentially an immunoassay except that the solid phase, instead of being a polystyrene microtiter plate, is a polystyrene paddle or dipstick. Reagents are the same and the format can either be simultaneous or sequential.

In a chromatographic strip test format, a capture antibody and a labeled antibody are dried onto a chromatographic strip, which is typically nitrocellulose or nylon of high porosity bonded to cellulose acetate. The capture antibody is usually spray dried as a line at one end of the strip. At this end there is an absorbent material that is in contact with the strip. At the other end of the strip the labeled antibody is deposited in a manner that prevents it from being absorbed into the membrane. Usually, the label attached to the antibody is a latex bead or colloidal gold. The assay may be initiated by applying the sample immediately in front of the labeled antibody.

Immunofiltration/immunoconcentration formats combine a large solid phase surface with directional flow of sample/reagents, which concentrates and accelerates the binding of antigen to antibody. In a preferred format, the test sample is preincubated with a labeled antibody then applied to a solid phase such as fiber filters or nitrocellulose membranes or the like. The solid phase can also be precoated with latex or glass beads coated with capture antibody. Detection of analyte is the same as standard immunoassay. The flow of sample/reagents can be modulated by either vacuum or the wicking action of an underlying absorbent material.

A threshold biosensor assay is a sensitive, instrumented assay amenable to screening large numbers of samples at low cost. In one embodiment, such an assay comprises the use of light addressable potentiometric sensors wherein the reaction involves the detection of a pH change due to binding of the desired protein by capture antibodies, bridging antibodies and urease-conjugated antibodies. Upon binding, a pH change is effected that is measurable by translation into electrical potential (μvolts). The assay typically occurs in a very small reaction volume, and is very sensitive. Moreover, the reported detection limit of the assay is 1,000 molecules of urease per minute.

The present invention also provides for probes and primers for detecting Streptococcus pneumoniae and Streptococcus pyogenes.

In one embodiment of this aspect of the invention, probes are provided that are capable of specifically hybridizing to S. pneumoniae and S. pyogenes Hsp60 genes DNA or RNA. For purposes of the present invention, probes are “capable of hybridizing” to S. pneumoniae and S. pyogenes Hsp60 genes DNA or RNA if they hybridize under conditions of high stringency (see Sambrook et al. (supra)). Preferably, the probe may be utilized to hybridize to suitable nucleotide sequences under highly stringent conditions, such as 6×SSC, 1× Denhardt's solution (Sambrook et al. (supra)), 0.1% SDS at 65° C. and at least one wash to remove excess probe in the presence of 0.2×SSC, 1×Denhardt's solution, 0.1% SDS at 65° C. Except as otherwise provided herein, probe sequences are designed to allow hybridization to Streptococcal DNA or RNA sequences, but not to DNA or RNA sequences from other organisms, particularly other bacterial sequences. The probes are used, for example, to hybridize to nucleic acid that has been exposed from a cell in a sample. The hybridized probe is then detected, thereby indicating the presence of the desired cellular nucleic acid. Preferably, the cellular nucleic acid is subjected to an amplification procedure, such as PCR, prior to hybridization.

Probes of the present invention may be composed of either deoxyribonucleic acids (DNA) or ribonucleic acids (RNA), and may be as few as about 12 nucleotides in length, usually about 14 to 18 nucleotides in length, and possibly as large as the entire sequence of the S. pneumoniae and S. pyogenes Hsp60 genes. Selection of probe size is somewhat dependent upon the use of the probe, and is within the skill of the art.

Suitable probes can be constructed and labeled using techniques that are well known in the art. Shorter probes of, for example, 12 bases can be generated synthetically. Longer probes of about 75 bases to less than 1.5 kb are preferably generated by, for example, PCR amplification in the presence of labeled precursors such as [α-³²P]dCTP, digoxigenin-dUTP, or biotin-dATP. Probes of more than 1.5 kb are generally most easily amplified by transfecting a cell with a plasmid containing the relevant probe, growing the transfected cell into large quantities, and purifying the relevant sequence from the transfected cells. (See Sambrook et al. (supra)).

Probes can be labeled by a variety of markers, including for example, radioactive markers, fluorescent markers, enzymatic markers, and chromogenic markers. The use of ³²P is particularly preferred for marking or labeling a particular probe.

It is a feature of this aspect of the invention that the probes can be utilized to detect the presence of S. pneumoniae and S. pyogenes Hsp60 mRNA or DNA within a sample. However, if the bacteria are present in only a limited number, then it may be beneficial to amplify the relevant sequence such that it may be more readily detected or obtained.

A variety of methods may be utilized in order to amplify a selected sequence, including, for example, RNA amplification (see Lizardi et al., Bio/Technology 6:1197-1202, 1988; Kramer et al., Nature 339:401-402, 1989; Lomeli et al., Clinical Chem. 35(9):1826-1831, 1989; U.S. Pat. No. 4,786,600), and DNA amplification utilizing LCR or Polymerase Chain Reaction (“PCR”) (see U.S. Pat. Nos. 4,683,195, 4,683,202, and 4,800,159; see also U.S. Pat. Nos. 4,876,187 and 5,011,769, which describe an alternative detection/amplification system comprising the use of scissile linkages), or other nucleic acid amplification procedures that are well within the level of ordinary skill in the art. With respect to PCR, for example, the method may be modified as known in the art. PCR may also be used in combination with reverse dot blot hybridization (Iida et al., FEMS Microbiol. Lett. 114:167-172, 1993). PCR products may be quantitatively analyzed by incorporation of dUTP (Duplàa et al., Anal. Biochem. 212:229-236, 1993), and samples may be filter sampled for PCR-gene probe detection (Bej et al., Appl. Environ. Microbiol. 57:3529-3534, 1991).

Within a preferred embodiment, PCR amplification is utilized to detect S. pneumoniae and S. pyogenes Hsp60 DNA. Briefly a DNA sample is denatured at 95° C. in order to generate single-stranded DNA. Specific primers are then annealed to the single-stranded DNA at 37° C. to 70° C., depending on the proportion of AT/GC in the primers. The primers are extended at 72° C. with Taq DNA polymerase in order to generate the opposite strand to the template. These steps constitute one cycle, which may be repeated in order to amplify the selected sequence.

Within an alternative preferred embodiment, LCR amplification is utilized for amplification. LCR primers are synthesized such that the 540 base of the upstream primer is capable of hybridizing to a unique base pair in a desired gene to specifically detect a strain of Streptococcus harboring the desired gene.

Within another preferred embodiment, the probes are used in an automated, non-isotopic strategy wherein target nucleic acid sequences are amplified by PCR, and then desired products are determined by a calorimetric oligonucleotide ligation assay (OLA) (Nickerson et al., Proc. Natl. Acad. Sci. USA 81:8923-8927, 1990).

Primers for the amplification of a selected sequence should be selected from sequences that are highly specific and form stable duplexes with the target sequence. The primers should also be non-complementary, especially at the 3′ end, should not form dimers with themselves or other primers, and should not form secondary structures or duplexes with other regions of DNA. In general, primers of about 18 to 20 nucleotides are preferred, and can be easily synthesized using techniques well known in the art. PCR products, and other nucleic acid amplification products, may be quantitated using techniques known in the art (Duplaa et al., Anal. Biochem. 212:229-236, 1993; Higuchi et al., Bio/Technology 11:1026-1030).

Further a biochip array specific for Streptococcus, comprised of a substrate to which either oligonucleotides or polypeptides may be bound can be manufactured using the invention disclosed herein in combination with current biochip technologies. U.S. Pat. No. 5,445,934. By using such a substrate with oligonucleotides derived from the Streptococcal Hsp60 sequences or antibodies specific for the Streptococcal gene products of this invention, a high throughput screening tool can be created to identify the specific Streptococcal strain in many samples.

E. Pharmaceutical Compositions and Methods

Another aspect of the present invention provides compositions and methods comprising one or more of the above-described Streptococcal Hsp60 polypeptides or antibodies to Streptococcal Hsp60 in combination with one or more pharmaceutically or physiologically acceptable carriers, adjuvants, binders or diluents. Such compositions can be used to elicit or enhance an immune response in a recipient animal, which is preferably a human being, and preferably elicits or enhances a protective or partially protective immunity against Streptococcus, or against an organism associated with an antigen fused to the Streptococcal Hsp60 s of the present invention.

Preferably, such carriers, adjuvants, binders or diluents are nontoxic to recipients at the dosages and concentrations employed. Ordinarily, the preparation of such compositions entails combining the isolated Streptococcal Hsp60 polypeptide with buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents. Examples of adjuvants include alum or aluminum hydroxide for humans.

It will be evident in light of the present specification to those in the art that the amount and frequency of administration can be optimized in clinical trials, and will depend upon such factors as the disease or disorder to be treated, the degree of immune inducement, enhancement, or protection required, and many other factors.

In one embodiment, the composition is administered orally, and the purified Streptococcal Hsp60 is taken up by cells, such as cells located in the lumen of the gut. Alternatively, the Streptococcal Hsp60 composition can be parenterally administrated via the subcutaneous route, or via other parenteral routes. Other routes include buccausublingual, rectal, nasal, topical (such as transdermal and ophthalmic), vaginal, pulmonary, intraarterial, intramuscular, intraperitoneal, intraocular, intranasal or intravenous, or indirectly. The Streptococcal Hsp60 compositions of the present invention can be prepared and administered as a liquid solution, or prepared as a solid form (e.g., lyophilized) which can be administered in solid form or resuspended in a solution in conjunction with administration.

Depending upon the application, quantities of injected Streptococcal Hsp60 in the composition will vary generally from about 0.1 μg to 1000 mg, typically from about 1 μg to 100 mg, preferably from about 10 μg to 10 mg, and preferably from about 100 μg to 1 mg, in combination with the physiologically acceptable carrier, binder or diluent. Booster immunizations can be given from 2-6 weeks later.

The pharmaceutical compositions of the present invention may be placed within containers, along with packaging material, preferably consumer-acceptable, which provides instructions regarding the use of such pharmaceutical compositions, to provide kits suitable for use within the present invention. Generally, such instructions will include a tangible expression describing the reagent concentration, as well as within certain embodiments, relative amounts of excipient ingredients or diluents (e.g., water, saline or PBS) which may be necessary to reconstitute the pharmaceutical composition.

The Hsp gene products of this invention may also be used as immunological carriers in conjugate vaccines. Hsps are beneficial carriers of antigens because, unlike other carriers, they do not have an immunosuppressive effect. See Barrios et al., Eur. J. Immunol. 22:1365-1372, 1992; Suzue and Young, in Stress-Inducible Cellular Responses 77:451-465, 1996 (edited by U. Feige et al.). Such carriers may be used to elicit an increased immune response to the conjugated molecule. The Streptococcal Hsp gene products of this invention may therefore be used as carriers (in conjugates or fusion proteins).

An additional aspect of the present invention is the use of the Streptococcal Hsp60 genes and gene products to treat and/or prevent tumors. The methods comprise administering to an individual having cancer a composition comprising a purified Streptococcal Hsp60 gene product as discussed herein in an amount effective to elicit and/or enhance the immune response of an individual against the cancer. The present invention also provides a method of immunizing an individual against cancer, or of providing at least a partially effective immunoprotective response in such an individual, the method comprising administering to the individual a composition comprising a purified Streptococcal Hsp60 as discussed herein in an amount effective to immunize the individual.

Preferably, the treatment of cancer comprises the use of highly purified Streptococcal Hsp60 gene products that are substantially free of endotoxins and methods and compositions related to the same. Such highly purified proteins are particularly advantageous, for example, for the treatment of human cancers because they do not incur the adverse side effects associated with such endotoxins. In particular, the compositions are capable of inducing an immune response against a cancer existing within an individual, which includes both eliciting the immune response or enhancing the immune response against the cancer. For example, the cancer to be treated may be an endothelial cell cancer, such as a sarcoma and/or breast, ovarian, prostate, lung, pancreas and liver cancers. The present invention also provides compositions that are capable of providing either partially or fully protective immune responses by immunization against cancers that are not yet present within an individual.

A further aspect of the present invention is protection from a variety of bacterial diseases by either immunization with the Hsp60 gene products of the present invention or by using gene transfer techniques to deliver a vector containing Streptococcal Hsp60 genes or fragments thereof to be expressed within the cells of the animal. The compositions and methods of the present invention can also provide for cancer prevention.

The compositions and methodologies described herein are suitable for a variety of uses. To this end, the following examples are presented for purposes of illustration, not limitation.

### EXAMPLES

### Example 1

### Isolation of Genes for Streptococcus pneumoniae and Streptococcus pyogenes Hsps

Genomic DNA from Streptococcus pneumoniae (ATCC6314) and Streptococcus pyogenes (ATCC12344), prepared by a routine method, was obtained from Dr. Lee Weber, University of Nevada at Reno.

Hsp60 DNA sequences were isolated by use of the polymerase chain reaction. Primers were designed based on N- and C-terminal homology of known Hsp60 sequences from other organisms. DNA amplifications of Streptococcal DNA were carried out using Taq polymerase (Perkin-Elmer). About 20 different primer pairs were tested using different reaction conditions. One pair (pair 1) was identified that was capable of amplifying Hsp60-1 genes, and a second (pair 2) that permitted amplification of Hsp60-2 sequences. Reaction mixtures capable of amplifying Hsp60 sequences contained, in a total volume of 50 μl, 0.5 μg of genomic DNA, 50 pmoles of each of a pair of degenerate primers, 500 μM each of dNTPs, 1×PCR buffer (Perkin-Elmer), 2 mM MgSO₄, and 1.25 units of Taq polymerase (Perkin-Elmer). The following two pairs of degenerate primers were employed successfully:

Pair 1:

| | |
|---|---|
| forward primer #1F: | |
| 5′-CATATGGCNGCNAAAGAYGTAAAA-3′ | (SEQ ID NO: 35) |
| reverse primer #1R: | |
| 5′-TGATCACATCATNCCNCCCATNCC-3′ | (SEQ ID NO: 36) |

Pair 2:

| | |
|---|---|
| forward primer #2F: | |
| 5′-CATATGGCAAAAGAAATHAARTTY-3′ | (SEQ ID NO: 37) |
| reverse primer #2R: | |
| 5′-TGATCANCCNCCCATNCCNCCCAT-3′ | (SEQ ID NO: 38) |

In the above sequences, N refers to A, C, G or T, and H to A, C or T (not G).

Reactions were cycled 35 times at 94° C. for 1 minute, 50° C. for 2 minutes and 72° C. for 2 minutes. PCR products were electrophoresed on 0.6% low-melting point agarose gels (Gibco-BRL) along with molecular weight markers. After staining with ethidium bromide, DNA fragments were visualized under low-intensity, long-wavelength UV illumination, and fragments of about 1.6 kbp were excised. DNA was isolated from gel slices by phenol extraction and ethanol precipitation (Maniatis et al.). Purified fragments were ligated to pCRII TA cloning vector (Invitrogen), and ligation mixtures were used to transform E. coli strain DH5a. (Competent cells obtained from Life Technologies.) Recombinant plasmids were isolated from kanamycin-resistant colonies by a standard alkaline lysis method, and the presence in plasmids of DNA inserts was verified by digestion with EcoRI digestion followed by agarose gel electrophoresis and visualization of digestion products by staining with ethidium bromide.

### Example 2

### Nucleotide Sequence Analysis of Streptococcal Hsp60

Inserts present in recombinant pCRII-based clones were sequenced using a CircumVent sequencing kit (New England Biolabs), ³⁵S-dATP and primers listed below. Multiple clones containing particular Streptococcal Hsp60 genes were sequenced: sequences were obtained from five clones, derived from three independent PCR reactions, of the Streptococcus pneumoniae Hsp60-1 gene, two clones, derived from single PCR reactions, of the Streptococcus pneumoniae Hsp60-2 gene, four clones, derived from three independent PCR reactions, of the Streptococcus pyogenes Hsp60-1 gene, and two clones and a portion of a third clone, derived from single PCR reaction, of the Streptococcus pyogenes Hsp60-2 gene. Sequencing reactions were fractionated on denaturing 6% polyacrylamide-8M urea gels (60 cm length), and the gels were dried and exposed for autoradiography. Autoradiographs were read manually, and sequence data were assembled and compared to other known Hsp60 genes using DNA Strider software (CEA, France).

Sequencing primers used:

| | | |
|---|---|---|
| M13F: | 5′-GTAAAACGACGGCCAG-3′ | (SEQ ID NO:39) |
| M13R: | 5′-CAGGAAACAGCTATGAC-3′ | (SEQ ID NO:40) |
| W178: | 5′-CCAACCATCACGAAAGA-3′ | (SEQ ID NO:41) |
| W179: | 5′-ACGGGTCACTTTGGTTG-3′ | (SEQ ID NO:42) |
| W189: | 5′-TTACTAATGACGGGGTA-3′ | (SEQ ID NO:43) |
| W190: | 5′-TTACCAATGACGGTGTG-3′ | (SEQ ID NO:44) |
| W191: | 5′-ACAGGGTCAATGATTCC-3′ | (SEQ ID NO:45) |
| W192: | 5′-ACTGGATCAATGATACC-3′ | (SEQ ID NO:46) |
| W195: | 5′-CCGTACCGTGCTCTGAC-3′ | (SEQ ID NO:47) |
| W196: | 5′-ACCACGTTTCAGATCCA-3′ | (SEQ ID NO:48) |
| W197: | 5′-GACAGTTTCGCGGCAAC-3′ | (SEQ ID NO:49) |
| W198: | 5′-CTCAGAACGAAGATCAG-3′ | (SEQ ID NO:50) |
| W200: | 5′-GGTATGCAGTTCGACCG-3′ | (SEQ ID NO:51) |
| W201: | 5′-CCGTGTTGGTCAAATCC-3′ | (SEQ ID NO:52) |
| W202: | 5′-GGTAACTACGGTTACAA-3′ | (SEQ ID NO:53) |
| W203: | 5′-GAGGCCACTTCTTTCAC-3′ | (SEQ ID NO:54) |
| W204: | 5′-GGCTTCCAGCACTGGCA-3′ | (SEQ ID NO:55) |
| W205: | 5′-AACTTCAGTCGCAGCAC-3′ | (SEQ ID NO:56) |
| W206: | 5′-CCTTGAAAGCCATTGCT-3′ | (SEQ ID NO:57) |
| W207: | 5′-GCTACACGTGCAGCCGT-3′ | (SEQ ID NO:58) |
| W208: | 5′-GCTGCAACAGGTGAGTG-3′ | (SEQ ID NO:59) |
| W209: | 5′-TCATGAACAATGGCTTG-3′ | (SEQ ID NO:60) |
| W210: | 5′-ACGAAGCACAATGTTAC-3′ | (SEQ ID NO:61) |
| W211: | 5′-ATCACTAAAGATGGTGT-3′ | (SEQ ID NO:62) |
| W214: | 5′-GCAGTTGCCGCAGCAGT-3′ | (SEQ ID NO:63) |
| W215: | 5′-GCTACTCGTGCAGCTGT-3′ | (SEQ ID NO:64) |
| W216: | 5′-GTTCTCCGTGCTTTGGA-3′ | (SEQ ID NO:65) |
| W217: | 5′-GCACCTGCTGTGACGTT-3′ | (SEQ ID NO:66) |
| W218: | 5′-TCTTCGATGGTGATGAC-3′ | (SEQ ID NO:67) |
| W219: | 5′-GGCAAGAGCTGTTCCGC-3′ | (SEQ ID NO:68) |
| W220: | 5′-CTGAGCCAGTACGGTTG-3′ | (SEQ ID NO:69) |
| W221: | 5′-GTACTGCAGAGCGGAAC-3′ | (SEQ ID NO:70) |
| W224: | 5′-ACCGTCTTCAACGGTGA-3′ | (SEQ ID NO:71) |
| W225: | 5′-GTTATCATTGCTGAAGA-3′ | (SEQ ID NO:72) |
| W226: | 5′-ACGGTACCGCCGGTCAG-3′ | (SEQ ID NO:73) |
| W227: | 5′-CTGGGCCAGGCTAAACG-3′ | (SEQ ID NO:74) |
| W228: | 5′-CGACTGAAGTTGAAATG-3′ | (SEQ ID NO:75) |
| W229: | 5′-GCTGTTGAAGAACTGAA-3′ | (SEQ ID NO:76) |
| W230: | 5′-GTCTTCAACGGTGATCA-3′ | (SEQ ID NO:77) |
| W232: | 5′-TCTTCTACCGCAGCACG-3′ | (SEQ ID NO:78) |
| W233: | 5′-CTCTTGATTATTGCGGA-3′ | (SEQ ID NO:79) |
| W234: | 5′-TTGTTCAAAACAAGAGT-3′ | (SEQ ID NO:80) |
| W235: | 5′-CGATTATTGTAGAAGGT-3′ | (SEQ ID NO:81) |
| W236: | 5′-CTTGATAACCGCAACAC-3′ | (SEQ ID NO:82) |
| W237: | 5′-TCCAAAGCACGGAGAAC-3′ | (SEQ ID NO:83) |
| W238: | 5′-GTGTCAAACATCCAAGA-3′ | (SEQ ID NO:84) |
| W239: | 5′-TCTTCGATGGTAATCAC-3′ | (SEQ ID NO:85) |
| W240: | 5′-GCAATAATGAGTAATGG-3′ | (SEQ ID NO:86) |
| W241: | 5′-ACAGTAATTGTTGAAGG-3′ | (SEQ ID NO:87) |
| W242: | 5′-CAGTGCAATACGGTTAG-3′ | (SEQ ID NO:88) |
| W243: | 5′-AGCTTCGAGAACCGGCA-3′ | (SEQ ID NO:89) |
| W244: | 5′-GTGATCATCGCTGAAGA-3′ | (SEQ ID NO:90) |
| W245: | 5′-ACGGTTATTGTAGAAG-3′ | (SEQ ID NO:91) |

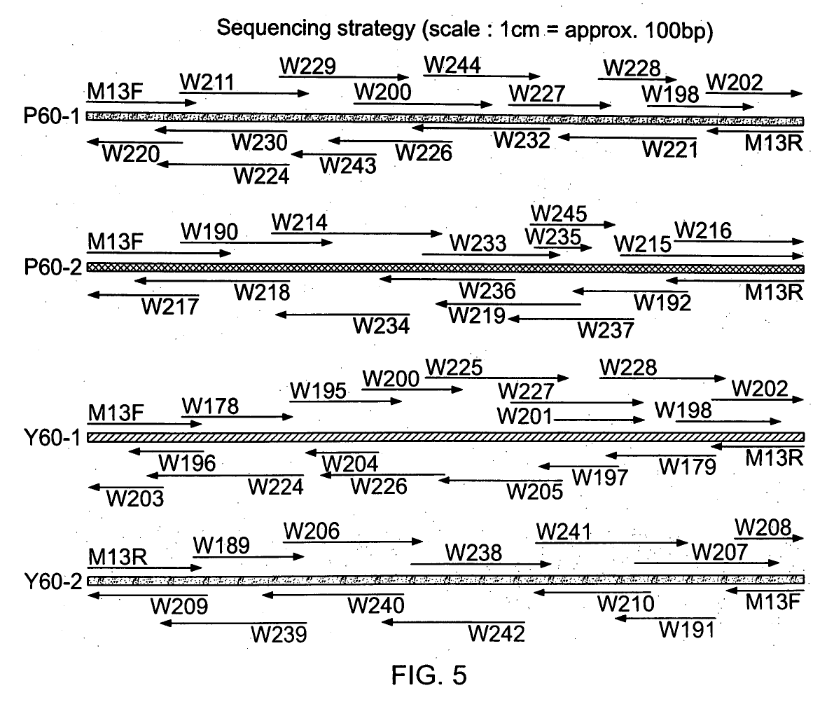

The sequencing strategy for each of the Hsp60 genes is set forth in FIG. 5. The nucleotide sequences of the Streptococcus pneumoniae Hsp60-1 gene (referred to as P60-1), the Streptococcus pneumoniae Hsp60-2 gene (P60-2), the Streptococcus pyogenes Hsp60-1 gene (Y60-1) and the Streptococcus pyogenes Hsp60-2 gene (Y60-2), and the corresponding deduced amino acid sequences, are set forth in FIGS. 1-4 (SEQ ID NOS: 1-8).

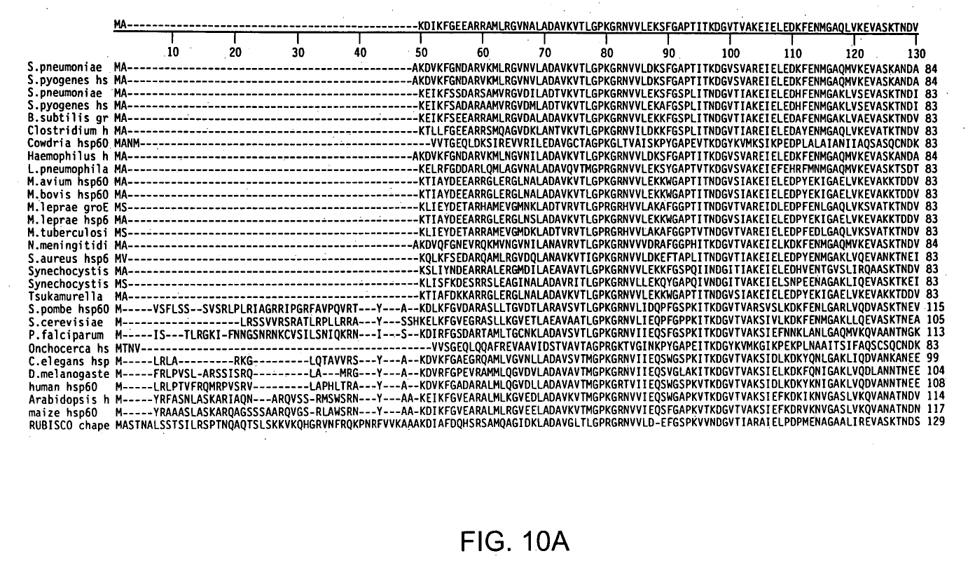

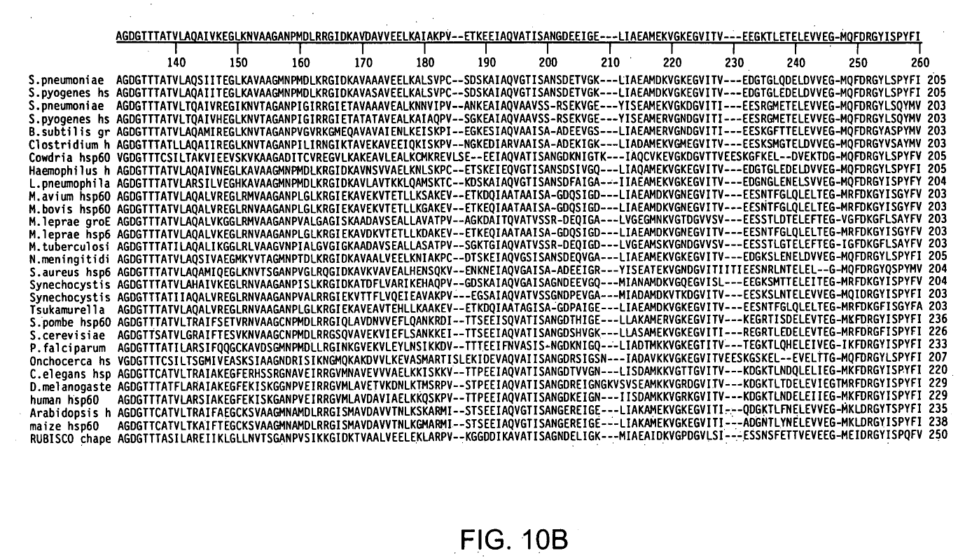

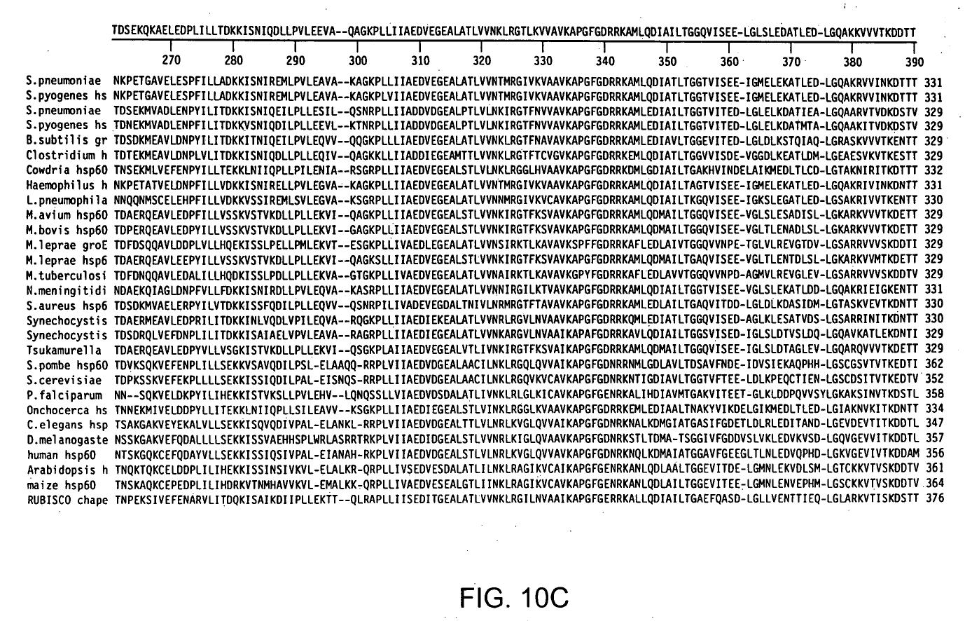

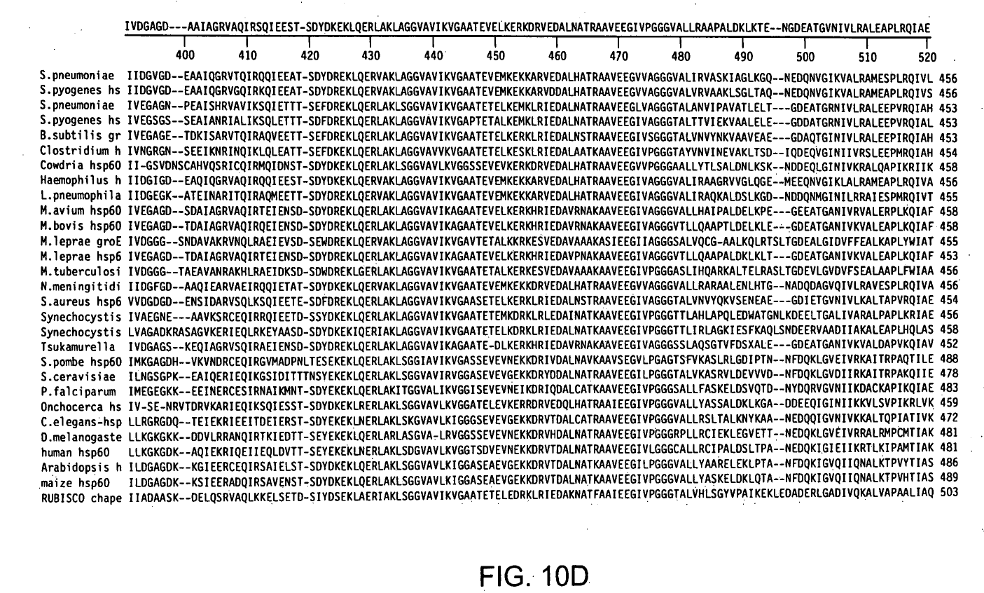

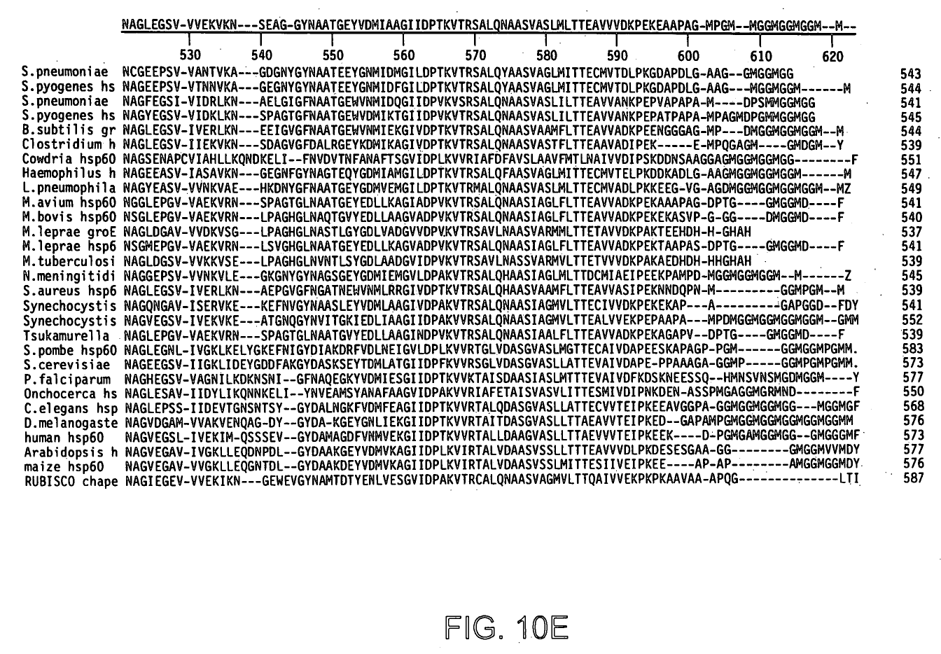

Comparisons of Streptococcal Hsp60 proteins and mycobacterial Hsp65 and GroEL proteins were determined using the MegAlign module of a DNA Star software package (DNASTAR, Inc.), and sequence similarities to Genbank-listed genes and proteins were uncovered using the BLAST algorithm (National Center for Biotechnology Information, NIH, Bethesda, Md.). One comparison of such sequences is set forth in FIG. 10.

### Example 3

### Expression of Recombinant Streptococcal Hsp60

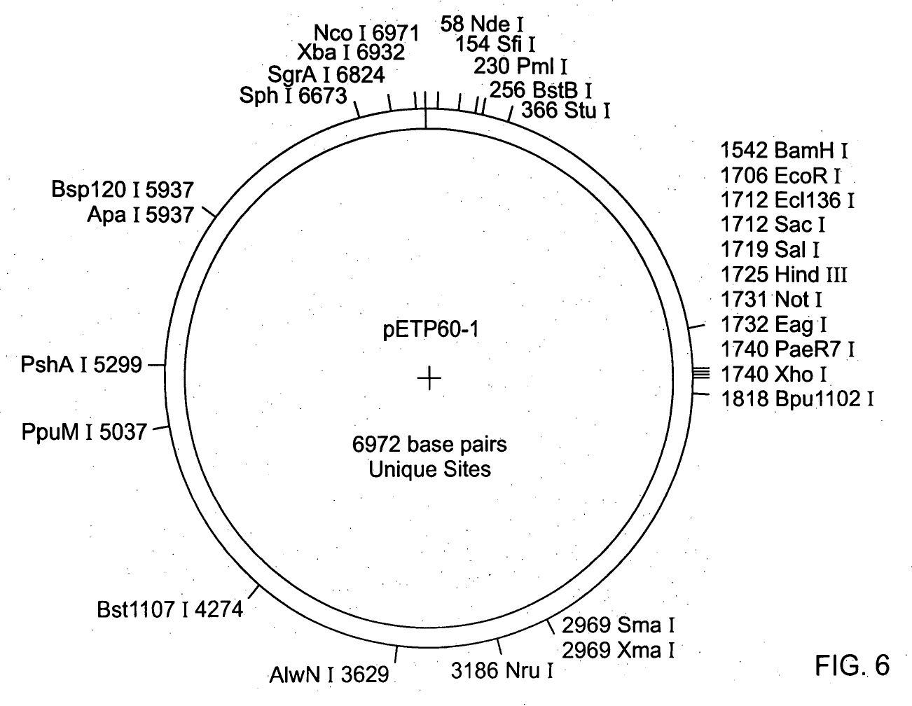

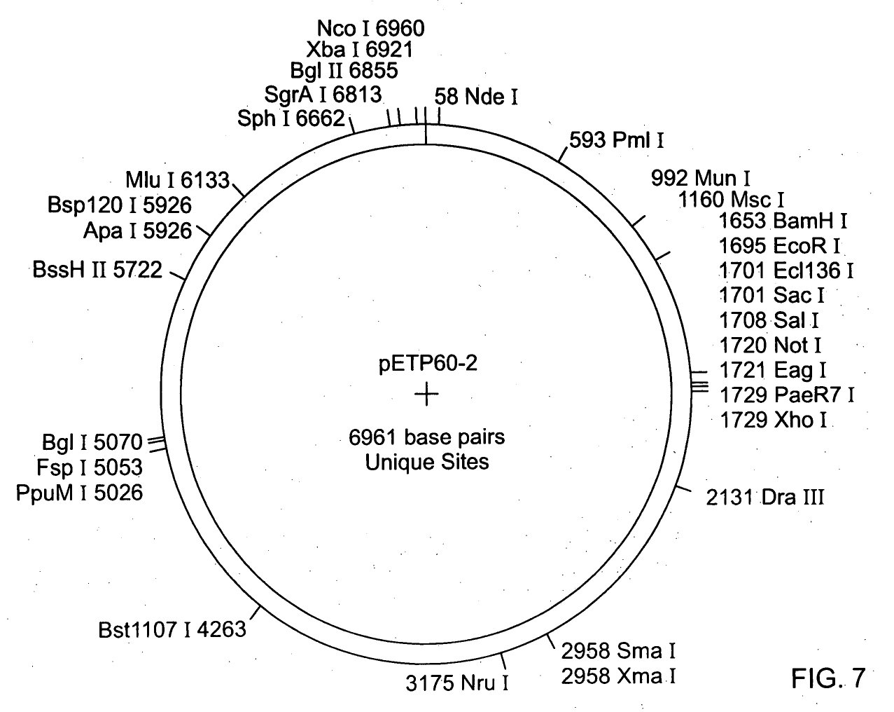

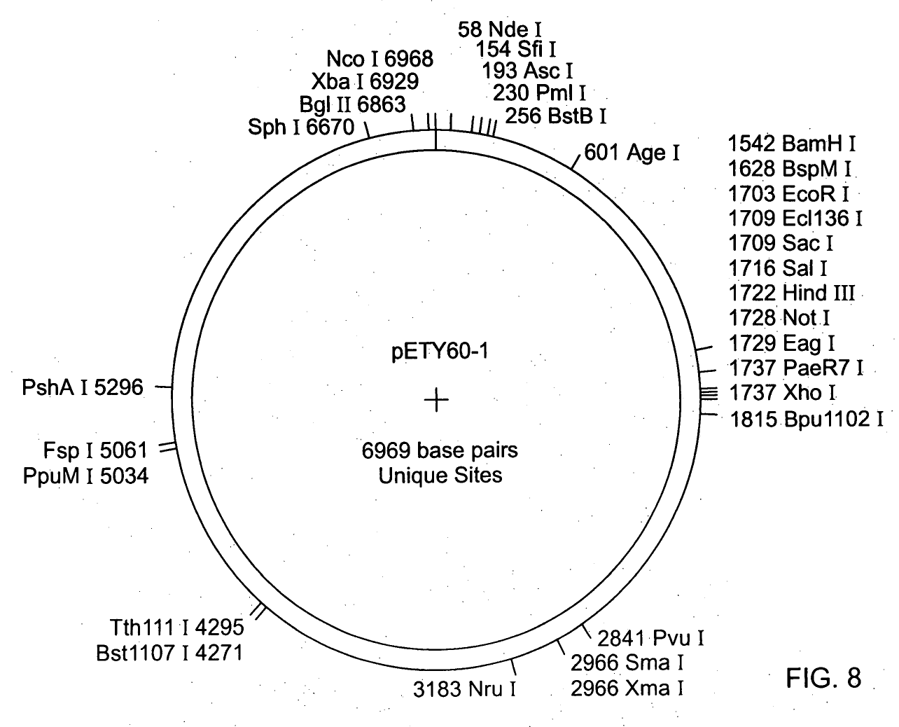

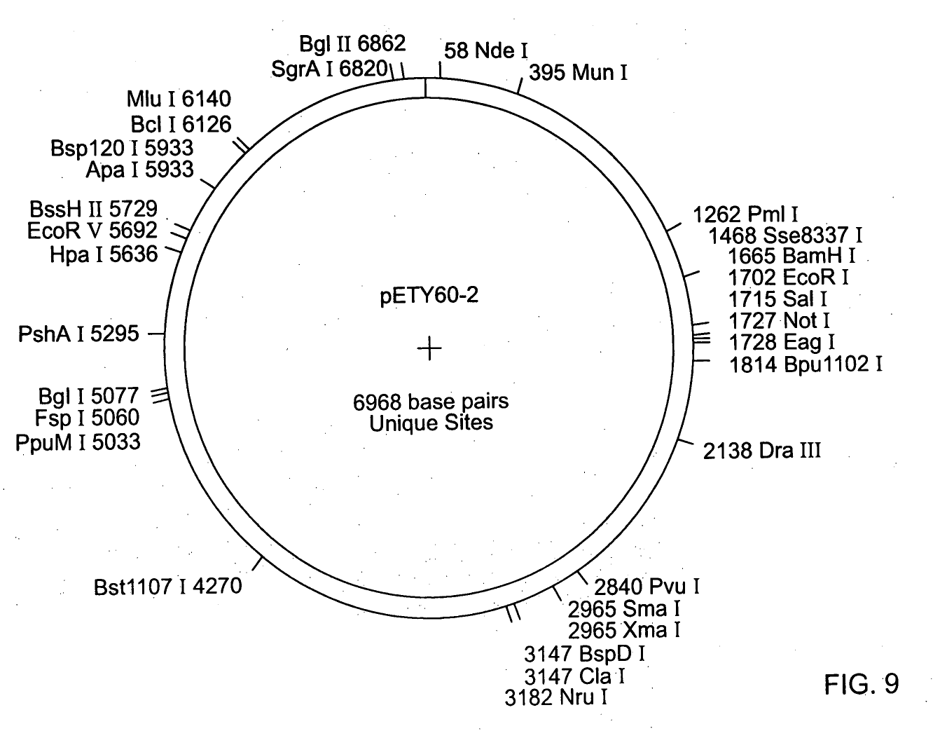

Inserts (Hsp60 genes) were excised from recombinant pCRII-based plasmids with restriction enzymes NdeI and EcoRI. NdeI cut inside forward PCR primers #1F or #2F, and EcoRI cut a short distance downstream from reverse PCR primers #1R or #2R in the polylinker region of vector PCRII. DNA fragments including Hsp60 gene sequences were fractionated on low-melting-point agarose gels, purified from the gels and ligated into NdeI/EcoRI double-digested pET28a(+) vector DNA (Novagen). Ligation reactions were used to transform competent Escherichia coli DH5a cells, and transformants were selected on Luria Broth plates containing 30 μg/ml of kanamycin D. DNA was isolated from single colonies using a standard alkaline lysis method, and the presence of correct inserts verified by digestion with NdeI and EcoRI and agarose gel electrophoresis. The resulting expression plasmids contained either a Streptococcus pneumoniae Hsp60-1 gene (referred to as pETP60-1), a Streptococcus pneumoniae Hsp60-2 gene (pETP60-2), a Streptococcus pyogenes Hsp60-1 gene (pETY60-1) or a Streptococcus pyogenes Hsp60-2 gene (pETY60-2). Schematic maps of the expression plasmids are shown in FIGS. 6-9.

To test whether they were capable of expressing the inserted Streptococcal Hsp60 genes, the expression plasmids were introduced into Escherichia coli strain BL21(DE3) by electroporation, and transformant colonies were selected on kanamycin-containing plates as before. Cultures of one ml were inoculated with single colonies, and transformants were grown at 37° C., until the cultures were turbid. After removing an aliquot for analysis of proteins prior to induction of recombinant genes (uninduced cultures), isopropyl-thio-galactopyranoside (IPTG) was added to 1 mM, and cultures were incubated for an additional one or two hours (induced cultures). Aliquots of 100 μl of induced and uninduced cultures were centrifuged at 12,000×g for 30 seconds. Bacterial pellets were lysed in 100 μl of SDS-PAGE loading buffer and boiled for 3 minutes. Aliquots of 10 μl of lysates were analyzed by 10% SDS-PAGE. Recombinant Streptococcal Hsp60 proteins were detectable after Coomasie blue staining as prominent bands migrating with an apparent molecular weight of about 60 kDa, which bands were present in induced but not in uninduced samples.

### Example 4

### Purification of Recombinant Streptococcal Hsp60

Bacteria containing recombinant Streptococcal Hsp60 expression plasmids were grown in 2×YT medium (20 g Tryptone, 10 g yeast extract, 10 g NaCl per liter) supplemented with 30 μg/ml of kanamycin D at 37° C. to an optical density at 600 nm of 0.5-0.8 and then induced with 0.5 mM IPTG for 3 hours. Cultures were then chilled on ice, and bacteria collected by centrifugation at 7,000×g for 5 min (at 4° C.). Bacterial pellets were frozen at −80° C.

Frozen bacterial pellet was crushed, transferred to a blender and homogenized in 200 ml of buffer A (6 M guanidinium hydrochloride, 50 mM Tris-HCl pH 7.5, 0.5 mM beta-mercaptoethanol).

Lysate was cleared by centrifugation at 10,000×g for 15 min (at 4° C.). The supernatant solution was mixed overnight at room temperature with approximately 100 ml of slurry containing 50 ml of Ni-SEPHAROSE (Chelating Sepharose, Pharmacia) equilibrated in buffer A. The resin was then washed on filter paper with approximately 200 ml buffer A, resuspended in small volume of the same buffer and gravity-packed into glass chromatography column (Pharmacia).

The column was washed with 20 ml of buffer A with 1% TRITON X-100. The column was further washed with a 6-0 M guanidinium hydrochloride/0-1 M NaCl gradient in 50 mM Tris-HCl pH 7.5, 0.5 mM beta-mercaptoethanol (200 ml), then with 200 ml of 50 mM Tris-HCl pH 7.5, 1 M NaCl, 0.5 mM beta-mercaptoethanol, and finally with 200 ml of 50 mM imidazole, 0.5 M NaCl, 50 mM Tris-HCl pH 7.5, 1.025 M NaCl, 0.5 mM beta-mercaptoethanol. Then column was developed with a 200 ml-gradient from 5% to 100% of buffer composed of 1 M imidazole, 0.5 M NaCl, 50 mM Tris-HCl pH 7.5, 0.5 mM beta-mercaptoethanol in 1M NaCl, 50 mM Tris-HCl pH 7.5, 0.5 mM beta-mercaptoethanol. Fractions of 9 ml were collected. The flow rate was 4-5 ml/min, and chromatography was monitored by absorbance at 280 nm.

Fractions containing the highest concentrations of recombinant protein were identified by 10% SDS-PAGE as before, pooled (usually 5-6 fractions) into a dialysis bag (12 kDa cutoff). Protein solution (approximately 50 ml) was then dialysed in the cold against three changes of 3 liters of Dulbeccos' phosphate-buffered saline (2.7 mM KH₂PO₄, 4.3 mM Na₂HPO₄, 2.7 mM KCl, 0.137 M NaCl). Dialysed protein was aliquoted and stored at −80° C. Usually 200-400 mg of recombinant protein were obtained (estimated by protein assay according to Lowry).

### Example 5

### Characterization of Purified, Recombinant Hsp60

To unambiguously identify recombinant proteins as Streptococcal Hsp60, purified recombinant proteins were subjected to

N- and C-terminal sequencing (conducted by the Protein Chemistry Facility, W. Alton Jones Cell Science Center, Lake Placid, N.Y.). These determinations revealed that purified recombinant proteins had the C- and N-terminal sequences predicted from the deducted amino acid sequences of SEQ ID NOS:5-8 (except for the N-terminal methionine that is typically processed away in E. coli bacteria).

### Example 6

### Reactivity of Recombinant Streptococcal Hsp60 with known anti-Hsp60 Monoclonal Antibodies

Purified recombinant Streptococcal Hsp60 proteins were analyzed for reactivity with the following commercially available antibodies:
A) Rabbit polyclonal antibody SPA-804 (StressGen Biotechnologies) which was raised against Synechococcus sp. Hsp60. The antibody recognizes Hsp60 from a wide range of prokaryotes and eukaryotes including cyanobacteria, Escherichia coli, and primate, murine, hamster, and rat cell lines.
B) Murine monoclonal antibody SPA-807 (StressGen Biotechnologies) which was raised against human Hsp60. Its epitope is located between residues 383-419 of that protein. The antibody also cross-reacts with Hsp60 from various other species including primates, rabbit, mouse, rat, hamster, Borrelia sp., Escherichia coli, Streptococcus pyogenes, Yersinia enterocolitica, Salmonella typhimurium, Treponema hyodysenteriae, Treponema innocense, Trichinella spiralis, yeast, and spinach chloroplasts.
C) Murine monoclonal antibody SPA-870 (StressGen Biotechnologies) which was raised against Escherichia coli GroEL. The antibody does not recognize eukaryotic Hsp60 proteins.
D) Murine polyclonal antibody which was raised against Mycobacterium tuberculosis BCG Hsp60 (StressGen Biotechnologies). The antibody does not cross-react with Escherichia coli groEL or eukaryotic Hsp60.
E) Murine monoclonal antibody recognizing recombinant histidine tag (Qiagen).

Samples containing 0.1 μg, 0.5 μg or 1 μg of recombinant protein were fractionated on 10% SDS-PAGE, and proteins were electroblotted onto nitrocellulose. Blots for analysis with antibodies SPA-804, SPA-807, SPA-870, and anti-BCG Hsp60 were blocked with 5% skim milk in PBS containing 0.05% TWEEN 20 overnight at room temperature. Blots were then incubated for one hour in the same buffer containing primary antibody (at a 1:1000 dilution except for anti-BCG Hsp60 antibody which was used at a 1:500 dilution). Blots were washed 3 times (10 min each) with PBS with 0.05% TWEEN 20 and incubated for an additional hour in PBS with 5% skim milk, 0.05% TWEEN 20 and goat anti-rabbit IgG—alkaline phosphatase (AP) conjugate (Sigma) or goat-anti-murine IgG—alkaline phosphatase (AP) conjugate (Sigma) (at 1:1000 dilutions), respectively. After 3 washes in PBS with 0.05% TWEEN 20 as before, blots were soaked in alkaline phosphatase reaction buffer (100 mM Tris-HCl (pH 9.5), 150 mM NaCl, 10 mM MgCl₂) and then developed in 0.05% nitroblue tetrazolium (NBT), 0.05% 5-bromo-4-chloro-3-indolyl phosphate (BCIP) in the same buffer, until signals were clearly visible (approximately 15 minutes).

A similar procedure was followed for anti-histidine tag antibody, except that blocking was in 3% bovine serum albumin in TBS (10 mM Tris-HCl, pH 7.5, 150 mM NaCl). Primary and secondary antibodies were diluted in TBS alone, and incubation with primary antibody (1:500 dilution) was for two hours. Washes were performed as follows: blots were first washed twice for 10 min in TBS containing 0.05% Tween 20 and 0.2% Triton X-100, and once for 10 min in TBS.

Recombinant histidine-tagged Hsp60 proteins were purified from overexpressing E. coli cells and probed on Western blot with polyclonal antibodies SPA-804 and anti-BCG Hsp60 as well as monoclonal antibodies SPA-870, SPA-807, and anti-histidine tag antibody. As is shown in Table 1, SPA-804 recognized all four Streptococcal Hsp60 proteins. In contrast, SPA-807 failed to crossreact with Streptococcus pneumoniae Hsp60-2, SPA-870 was unable to react with any Streptococcal Hsp60-2 protein, and anti-BCG Hsp60 failed to crossreact with any Streptococcal Hsp60. As predicted, anti-His tag antibody reacted with all recombinant proteins which had been expressed as His-tagged proteins. Positive reactivity is indicated as “+” while lack of it is marked with “−”.

TABLE 1
RECOGNITION OF STREPTOCOCCAL HSP60 PROTEINS
BY ANTI-HSP60 ANTIBODIES

| | S. pneumoniae | S. pneumoniae | S. pyogenes | S. pyogenes |
|---|---|---|---|---|
| Antibody | Hsp60-1 | Hsp60-2 | Hsp60-1 | Hsp60-2 |
| SPA-804 | + | + | + | + |
| SPA-807 | + | − | + | + |
| SPA-870 | + | − | + | − |
| anti- | − | − | − | − |
| BCG60 | | | | |
| anti-His | + | + | + | + |
| tag | | | | |

These data demonstrate that Streptococcal Hsp60 are antigenically distinct from Hsp60 of other organisms. They also show that Streptococcal Hsp60-1 and Hsp60-2 can be distinguished. And, they provide evidence that related Hsp60s from two different Streptococcal species can be recognized differentially by an antibody.

### Example 7

### Preparation and Identification of Peptide Fragments of Recombinant Streptococcal Hsp60

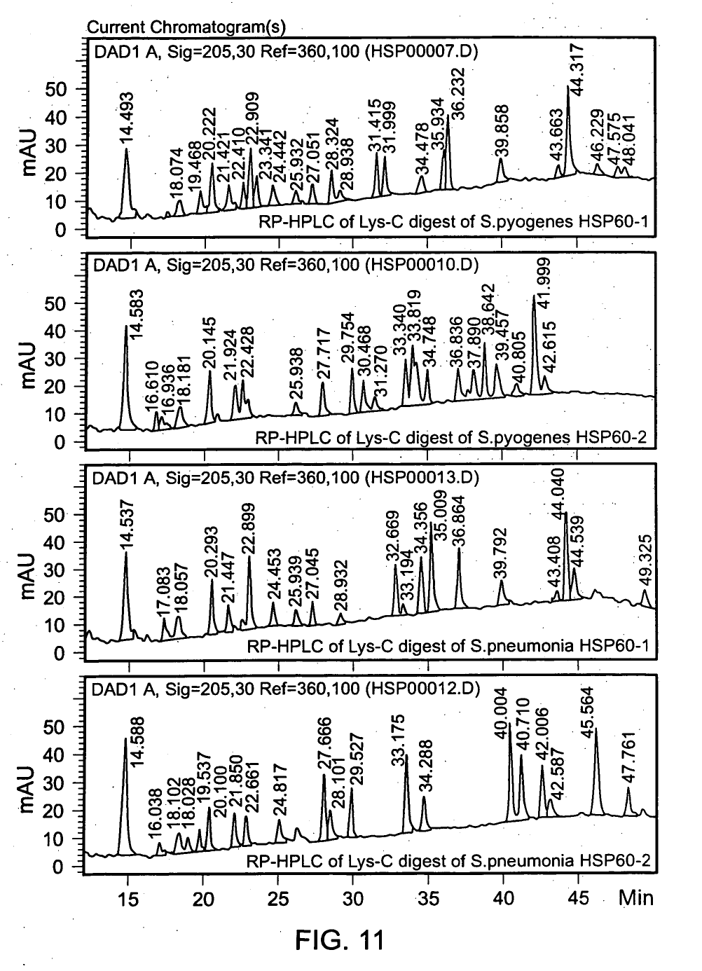

Purified recombinant proteins (50 mg at 1 mg/ml) were digested with 2.5 mg of Lys-C endopeptidase (Boehringer Mannheim) for 1 hour at 37° C. Digestion reactions were fractionated by capillary electrophoresis (3D-HPCE instrument, Hewlett-Packard). Reactions were run at 15 kV through a 75 u bare fused silica capillary in 50 mM dibasic sodium phosphate (pH 7.47). Alternatively, reverse phase chromatography (1100 Series HPLC instrument, Hewlett-Packard) was carried out on a Hamilton PRP-1 5 m column developed in a 0-60% acetonitrile gradient in water in the presence of 0.1% trifluoroacetic acid. Individual RP-HPLC-separated peptides of Hsp60 proteins were identified by mass spectroscopy by Hewlett-Packard Laboratories, Palo Alto, Calif. RP-HPLC chromatograms of digests of recombinant Streptoccocal Hsp60s are shown in FIG. 11.

### Example 8

### Identification of Endogenous Streptococcal Hsp60

Total protein extracts from Streptococcus pneumoniae (ATCC6314) and Streptococcus pyogenes (ATCC12344) were obtained from Dr. Lee Weber (University of Nevada, Reno). Equivalent amounts of both extracts (equalized based on intensity of staining of protein bands in SDS-PAGE gels) were fractionated by 10% SDS-PAGE alongside 50 ng of purified BGC Hsp60 (StressGen Biotechnologies). After electrotransfer onto nitrocellulose, filters were blocked, probed with antibody SPA-804, and antibody signals detected as described in Example 6.

Other, similarly prepared filters were incubated with a 1:3000 dilutions of antibodies SPA-807 or SPA-870 for one hour. Blots were rinsed twice with water, washed 3 times (5 min each) with PBS containing 0.05% TWEEN 20 and then incubated for an additional hour in PBS containing 5% skim milk, 0.05% TWEEN 20 and a 1:3000 dilution of goat anti-rabbit IgG—horseradish peroxidase (HRP) conjugate (Sigma). Subsequently, filters were rinsed with water, washed with PBS containing 0.05% TWEEN 20 as before, equilibrated in ECL substrate mixture (Amersham), wrapped in plastic wrap and exposed to X-ray film for between 15 seconds and 20 minutes.

The results from these experiments are summarized in Table 2. Antibody SPA-804 reacted strongly with both Streptococcal extracts. In contrast, antibody SPA-807 reacted weakly with extract from Streptococcus pneumoniae but strongly with extract from Streptococcus pyogenes. Finally, antibody SPA-870 reacted weakly with both Streptococcal extracts. Based on the antibody specificity determined in Example 6 (Table 1), it is concluded that Hsp60-2 is abundant in Streptococcal cells, whereas Hsp60-1 is only expressed at low levels. Presumably, Hsp60-1 is the more highly stress-inducible Hsp60 protein.

TABLE 2
REACTIVITY OF SELECTED ANTI-HSP60 ANTIBODIES WITH
PROTEIN EXTRACTS FROM S. PNEUMONIAE AND S. PYOGENES

| | BCG60 control | S. pneumoniae | S. pyogenes |
|---|---|---|---|
| Antibody | (50 ng) | extract | extract |
| SPA-804 | +++ | +++ | +++ |
| SPA-807 | +++ | + | +++ |
| SPA-870 | − | + | + a |

a: SPA870 detected protein with mobility different from predominant heavy band visualized in that extract with SPA-807. However, its mobility was close to the band detected in S. pneumoniae extract with both SPA-870 and SPA-807 antibodies.

The amount of the utilized extracts was normalized by comparing Coomassie stained gels containing serial dilutions.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. An isolated nucleic acid molecule encoding a Streptococcus pneumoniae Hsp60.

2. An isolated nucleic acid molecule encoding a Streptococcus pyogenes Hsp60 .

3. An isolated nucleotide molecule selected from the group consisting of:
(a) an isolated nucleic acid molecule comprising the sequence of SEQ ID NO: 1 from nucleotides 15-1652;
(b) an isolated nucleic acid molecule comprising the sequence of SEQ ID NO: 3 from nucleotides 15-1640;
(c) an isolated nucleic acid molecule comprising the sequence of SEQ ID NO: 5 from nucleotides 15-1649;
(d) an isolated nucleic acid molecule comprising the sequence of SEQ ID NO: 7 from nucleotides 15-1652;
(e) an isolated nucleic acid molecule complementary to any one of the nucleotides of SEQ ID NOS: 1, 3,5 or 7 set forth in (a) through (d), respectively; and
(f) an isolated nucleic acid molecule that hybridizes under conditions of high stringency to the nucleic acid molecules of any one of (a) through (e).

4-6. (canceled)

7. An isolated nucleic acid molecule according to claim 3, encoding a polypeptide that is able to be selectively bound by an antibody specific for a Streptococcus pneumoniae Hsp60 or a Streptococcus pyogenes Hsp60.

8-18. (canceled)

19. A vector comprising an isolated nucleic acid molecule according to claim 1.

20. The vector according to claim 19 wherein the vector is an expression vector comprising a promoter in operative linkage with the isolated nucleic acid molecule encoding the Hsp60 or portion thereof.

21. The vector according to claim 20, further comprising a selectable or identifiable marker.

22. The vector according claim 20 wherein the promoter is a constitutive or an inducible promoter.

23. A host cell containing a vector according to claim 19.

24. The host cell according to claim 23, wherein the host cell is selected from the group consisting of a bacterial cell, a mammalian cell, a yeast cell and an insect cell.

25-31. (canceled)
